(19) United States
(12) Patent Application Publication
(10) Pub. No.: US 2020/0172975 A1
(43) Pub. Date: Jun. 4, 2020

(54) Cell-Free microRNA Signatures of Pancreatic Islet Beta Cell Death

(71) Applicant: THE UNIVERSITY OF SYDNEY, SYDNEY (AU)

(72) Inventors: ANANDWARDHAN AWADHOOT HARDIKAR, SYDNEY (AU); MUGDHA VINAY JOGLEKAR, SYDNEY (AU); ANDRZEJ SZCZESNY JANUSZEWSKI, SYDNEY (AU)

(21) Appl. No.: 16/623,752

(22) PCT Filed: Jun. 22, 2018
(86) PCT No.: PCT/AU2018/000106

§ 371 Date: Dec. 17, 2019

(30) Foreign Application Priority Data
Jun. 29, 2017 (AU) 2017902521

(51) Int. Cl. C12Q 1/6883

(52) U.S. Cl. CPC C12Q 1/6883; C12Q 2600/178; G01N 2800/52; G01N 2800/50; G01N 2800/042

(57) ABSTRACT

The present invention relates generally to microRNA signatures associated with aberrant insulin production. The microRNA signatures are relevant, for example, to predicting, diagnosing and/or prognosing diseases and conditions associated with aberrant insulin production.

### INCORPORATION BY CROSS-REFERENCE

The present application claims priority from Australian provisional application number 2017902521 filed on 29 Jun. 2017, the entire contents of which are incorporated herein by cross-reference.

### TECHNICAL FIELD

The present invention relates generally to the field of medicine, and more specifically to insulin-related diseases and conditions. Described herein are microRNA signatures associated with aberrant insulin production. The microRNA signatures are relevant, for example, to predicting, diagnosing and/or prognosing diseases and conditions associated with aberrant insulin production.

### BACKGROUND

Insulin is a hormone generated in the pancreas. Clusters of cells in the pancreas, known as the islets of Langerhans, contain beta cells that make insulin and release it into the circulation. Insulin plays a major role in metabolism, assisting cells throughout the body to absorb glucose and use it for energy. For example, it lowers blood glucose levels by assisting muscle, fat, and liver cells absorb glucose from the bloodstream, it stimulates the liver and muscle tissue to store excess glucose (glycogen), and it lowers blood glucose levels by reducing glucose production in the liver.

There are numerous diseases and conditions arising from or associated with aberrant insulin production and/or abnormal insulin metabolism in humans including, for example, diabetes (e.g. Type 1, Type 2, gestational and others), hypoglycemia, insulinoma, metabolic syndrome, and obese hypertension. Diabetes is a chronic disease that occurs either when the body cannot effectively use the insulin it produces (Type 2 diabetes) or when the pancreas does not produce enough insulin (Type 1 diabetes). Hyperglycaemia, as well as the more life-threatening hypoglycemia are common outcomes of uncontrolled diabetes and over time can lead to serious damage to nerves, blood vessels, heart, eyes, and kidneys. The World Health Organization (WHO) and the International Diabetes Federation (IDF) estimate that more than 420 million adults are currently suffering from diabetes and its global prevalence has risen considerably in recent years.

Standard clinical tests for diagnosing abnormalities in the production of insulin typically rely on measurements of glucose in the blood (e.g. AIC, FPG and OGTT tests). However, these tests can be unreliable in view of other factors affecting glucose levels which are not associated with aberrant insulin production. Additionally, current tests can be time-consuming in that some require the monitoring of glucose levels over a time period. Expense, inaccuracies in measurement, and/or poor standardisation are other issues that associated with glucose-based tests for abnormalities in insulin production. Blood glucose tests also lack predictive power due to the capacity of pancreatic β-cells to produce the desired level of insulin even when the majority (>70%) of the β-cells are dead/dying. Insulin and c-peptide tests are also used to assess insulin levels. Insulin testing reflects endogenous and exogenous insulin, while C-peptide gives indication of only endogenous insulin. These tests may be used in isolation, but are often combined with (or used following) glucose testing. Apart from more significant cost, they share at least some of the disadvantages associated with glucose testing. In the case of Type 1 diabetes, genetic testing and measurement of autoantibodies are commonly used diagnostic and prognostic tools. However, such tests do not offer the desired predictive power for stratifying at-risk of Type 1 diabetes individuals to progressors and non-progressors.

A need exists for improved methods for predicting, diagnosing and/or prognosing diseases and conditions associated with abnormal (e.g. reduced) insulin production.

### SUMMARY OF THE INVENTION

The present invention alleviates at least one of the problems associated with current methods for diagnosing aberrant insulin production. This is facilitated by the detection and characterisation of circulating (i.e. extracellular) microRNA signatures indicative of beta-cell insulin production capacity. More specifically, the microRNA signatures disclosed herein may be used to detect the loss of insulin-producing pancreatic islet beta-cells. Without limitation, the microRNA signatures described herein may be used: (i) as biomarkers to inform for predicting, diagnosing, and/or prognosing the development of diseases and conditions associated with aberrant (e.g. reduced) insulin production (e.g. Type I diabetes); (ii) to monitor responses to interventions such as islet transplantation, vaccines and drugs aiming to retard β-cell loss; (iii) to select for treatments that can block β-cell death; and/or (iv) to guide the development of new treatments and therapies so as to lessen the burden of diseases and conditions associated with aberrant (e.g. reduced) insulin production.

In general, the intracellular microRNA signatures of the present invention may be biomarkers of pancreatic beta-cell death and can be used as candidates on any molecular diagnostic or equivalent platform in any human tissue or biofluids for prediction of diabetes progression.

The present invention relates at least in part to the following embodiments:

### Embodiment 1

A method for diagnosing, prognosing, or determining a likelihood of developing a disease or condition associated with or arising from reduced insulin production in a subject, the method comprising:

determining expression levels of ten or more extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject; and

comparing expression levels the microRNAs in the biological fluid sample to control expression level/s of the microRNAs generated from control biological fluid/s equivalent to the sample,

wherein elevated expression levels of the microRNAs in the biological fluid sample compared to the expression level/s of the microRNAs generated from the control biological fluid/s is indicative of a deficient or absent capacity for insulin production in beta-islet cells of the subject, and the reduced or absent insulin production in the beta-islet cells is:

(i) predictive that the subject will develop the disease or condition; or

(ii) diagnostic of the disease or condition in the subject; or

(iii) prognostic that the disease or condition will progress in the subject.

The ten or more extracellular microRNAs may be any combination of the listed microRNAs, provided that the combination comprises ten or more of the microRNAs.

### Embodiment 2

A method for monitoring the response of a subject to a treatment for a disease or condition associated with or arising from reduced insulin production in a subject, the method comprising:

determining expression levels of ten or more extracellular microRNAs selected from the group consisting of: miR-24, miR-26a, miR-146a, mmiR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject; and

comparing expression levels the microRNAs in the biological fluid sample to control expression level/s of the microRNAs generated from control biological fluid/s equivalent to the sample,

wherein elevated expression levels of the microRNAs in the biological fluid sample compared to the expression level/s of the microRNAs generated from the control biological fluid/s is indicative of deficient or absent capacity for insulin production in beta islet cells of the subject, and a deficient or absent response to the treatment by the subject.

The ten or more extracellular microRNAs may be any combination of the listed microRNAs, provided that the combination comprises ten or more of the microRNAs.

### Embodiment 3

The method of embodiment 2, wherein the treatment comprises beta-islet cell transplantation, and/or the administration of a vaccine or therapeutic drug designed to retard loss of beta-islet cells in the subject and/or loss of insulin production capacity in beta-islet cells of the subject.

### Embodiment 4

A method for assessing the efficacy of a treatment for inhibiting or preventing loss of beta-islet cell insulin production function and/or beta-islet cell death, the method comprising:

administering the treatment to a subject in need thereof,

determining expression levels of ten or more extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject; and

comparing expression levels the microRNAs in the biological fluid sample to control expression level/s of the microRNAs generated from control biological fluid/s equivalent to the sample,

wherein equivalent or reduced expression levels of the microRNAs in the biological fluid sample compared to the expression level/s of the microRNAs generated from the control biological fluid/s is indicative that the treatment has efficacy in inhibiting or preventing loss of beta-islet cell insulin production function and/or beta-islet cell death.

The ten or more extracellular microRNAs may be any combination of the listed microRNAs, provided that the combination comprises ten or more of the microRNAs.

### Embodiment 5

The method of any one of embodiments 1 to 4, further comprising removing any cells comprising nuclear material present in the biological fluid prior to determining expression levels of one or more extracellular microRNAs.

### Embodiment 6

The method of embodiment 5, wherein said removing of the cells comprising nuclear material comprises lysing less than: 10%, 5%, 4%, 3%, 2%, 1%, 0.5%, 0.2% or 0.1% of the cells.

### Embodiment 7

The method of any one of embodiments 1 to 6, wherein the condition or disease is selected from any of diabetes, pancreatitis, insulinoma, and pancreatic cancer.

### Embodiment 8

The method of embodiment 7, wherein the disease is diabetes.

### Embodiment 9

The method of embodiment 8, wherein the diabetes is Type 1 diabetes.

### Embodiment 10

The method of any one of embodiments 1 to 9, wherein the biological fluid sample is whole blood.

### Embodiment 11

The method of any one of embodiments 1 to 9, wherein the biological fluid sample is serum or plasma.

### Embodiment 12

The method of any one of embodiments 1 to 11, further comprising an initial step of obtaining the biological fluid sample from the subject.

### Embodiment 13

The method of any one of embodiments 1 to 12, wherein the ten or more microRNAs comprise or consist of any: 10, 11, 12, 13, 14, 15, 16, 17, or 18; of said microRNAs.

### Embodiment 14

The method of any one of embodiments 1 to 13, wherein the subject is of Australian ethnicity, of Chinese ethnicity, or of Indian ethnicity.

### Embodiment 15

The method of any one of embodiments 1 to 14, wherein the ten or more microRNAs are selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

### Embodiment 16

The method of embodiment 15, wherein the microRNAs comprise or consist of any: 10, 11, 12, 13, 14, 15, 16, 17, 18, 19, 20, 21, 22, or 23; of said microRNAs.

### Embodiment 17

The method of embodiment 15 or embodiment 16, wherein the subject is of Australian ethnicity or of Chinese ethnicity.

### Embodiment 18

The method of any one of embodiments 1 to 13, wherein the ten or more microRNAs are selected from the group consisting of: miR-16, miR-20a, miR-21, miR-24, miR-25, miR-26a, miR-26b, miR-27a, miR-30a-5p, miR-30b, miR-30c, miR-92a, miR-93, miR-126, miR-146a, miR-186, miR-199a-3p, miR-222, miR-223, and miR-340.

### Embodiment 19

The method of embodiment 18, wherein the microRNAs comprise or consist of any: 10, 11, 12, 13, 14, 15, 16, 17, 18, 19, or 20; of said microRNAs.

### Embodiment 20

The method of embodiment 18 or embodiment 19, wherein the subject is of Australian ethnicity or of Indian ethnicity.

### Embodiment 21

The method of any one of embodiments 1 to 13, wherein the ten or more microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

### Embodiment 22

The method of embodiment 21, wherein the microRNAs comprise or consist of any: 10, 11, 12, 13, 14, 15, 16, 17, 18, or 19; of said microRNAs.

### Embodiment 23

The method of embodiment 21 or embodiment 22, wherein the subject is of Chinese ethnicity or of Indian ethnicity.

### Embodiment 24

The method of any one of embodiments 1 to 13, wherein the ten or more microRNAs are selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223 Embodiment 25. The method of embodiment 24, wherein the microRNAs comprise or consist of any: 10, 11, 12, 13, 14, 15, 16, 17, 18, 19, 20, 21, 22, 23, 24, 25, 26, 27, 28 or 29; of said microRNAs.

### Embodiment 26

The method of embodiment 24 or embodiment 25, wherein the subject is of Australian ethnicity.

### Embodiment 27

The method of any one of embodiments 1 to 13, wherein the ten or more microRNAs are selected from the group consisting of: let-7e, miR-7, miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

### Embodiment 28

The method of embodiment 27, wherein the microRNAs comprise or consist of any: 10, 11, 12, 13, 14, 15, 16, 17, 18, 19, 20, 21, 22, 23, 24, 25, 26, 27, 28 or 29; of said microRNAs.

### Embodiment 29

The method of embodiment 27 or embodiment 28, wherein the subject is of Chinese ethnicity.

### Embodiment 30

The method of any one of embodiments 1 to 29, wherein the control biological fluid/s is/are obtained from control subject/s determined not to have a disease or condition associated with aberrant insulin production and/or abnormal insulin metabolism.

### Embodiment 31

The method of embodiment 30, wherein the control subject/s are a population of individuals determined not to have a disease or condition associated with aberrant insulin production and/or abnormal insulin metabolism.

### Embodiment 32

The method of embodiment 31 or embodiment 32, wherein the control subject/s do not have a disease or condition selected from the group consisting of diabetes, pancreatitis, insulinoma, and pancreatic cancer.

### Embodiment 33

A kit comprising primers, probes and/or other binding agents for use in detecting expression of at least ten microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject.

The at least ten microRNAs may be any combination of the listed microRNAs, provided that the combination comprises ten or more of the microRNAs.

### Embodiment 34

A microRNA signature comprising least ten microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93.

The at least ten microRNAs may be any combination of the listed microRNAs, provided that the combination comprises ten or more of the microRNAs.

### Embodiment 35

The kit of embodiment 33 or the microRNA signature of embodiment 34, wherein the at least ten microRNAs comprise or consist of any: 10, 11, 12, 13, 14, 15, 16, 17, or 18; of said microRNAs.

### Embodiment 36

The kit of embodiment 33 or embodiment 35, or the microRNA signature of embodiment 34 or embodiment 35, wherein the at least ten microRNAs are selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

### Embodiment 37

The kit or microRNA signature of embodiment 36, wherein the microRNAs comprise or consist of any: 10, 11, 12, 13, 14, 15, 16, 17, 18, 19, 20, 21, 22, or 23; of said microRNAs.

### Embodiment 38

The kit of embodiment 33 or embodiment 35, or the microRNA signature of embodiment 34 or embodiment 35, wherein the at least ten microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-146a, miR-186, miR-199a-3p, miR-222, miR-223, miR-340 and miR-27a.

### Embodiment 39

The kit or microRNA signature of embodiment 38, wherein the microRNAs comprise or consist of any: 10, 11, 12, 13, 14, 15, 16, 17, 18, 19, or 20; of said microRNAs.

### Embodiment 40

The kit of embodiment 33 or embodiment 35, or the microRNA signature of embodiment 34 or embodiment 35, wherein the at least ten microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

### Embodiment 41

The kit or microRNA signature of embodiment 40, wherein the microRNAs comprise or consist of any: 10, 11, 12, 13, 14, 15, 16, 17, 18, or 19; of said microRNAs.

### Embodiment 42

The kit of embodiment 33 or embodiment 35, or the microRNA signature of embodiment 34 or embodiment 35, wherein the at least ten microRNAs are selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223.

### Embodiment 43

The kit or microRNA signature of embodiment 42, wherein the microRNAs comprise or consist of any: 10, 11, 12, 13, 14, 15, 16, 17, 18, 19, 20, 21, 22, 23, 24, 25, 26, 27, 28 or 29; of said microRNAs.

### Embodiment 44

The kit of embodiment 33 or embodiment 35, or the microRNA signature of embodiment 34 or embodiment 35, wherein the at least ten microRNAs are selected from the group consisting of: let-7e, miR-7, miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

### Embodiment 45

The kit or microRNA signature of embodiment 44, wherein the microRNAs comprise or consist of any: 10, 11, 12, 13, 14, 15, 16, 17, 18, 19, 20, 21, 22, 23, 24, 25, 26, 27, 28 or 29; of said microRNAs.

### Embodiment 46

The kit of any one of embodiments 33 or 35 to 45, further comprising any one or more of:
(i) one or more reagents for extracting the biological fluid sample;
(ii) one or more reagents for reverse-transcribing RNA into cDNA;
(iii) one or more reagents for amplifying the cDNA;
(iv) control standards for normalisation of microRNA signature expression data and/or comparison of microRNA signature expression data to determine whether expression of the microRNA signature is increased, reduced, and/or within a standard range;
(v) any one of more of: buffers, washing reagents, RNAse inhibitors, precipitators (e.g. glycogen), nuclease-free water, salt solutions.

### Embodiment 47

Use of the kit of any one of embodiments 33 or 35 to 46, or the microRNA signature of any one of embodiments embodiment 34 to 46, for diagnosing, prognosing, or determining a likelihood of developing a disease or condition associated with or arising from reduced insulin production in a subject, wherein the disease or condition is selected from the group consisting of diabetes, pancreatitis, insulinoma, and pancreatic cancer.

### Embodiment 48

Use of one or more agents for determining expression levels of ten or more extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from a subject, for the preparation of a medicament for diagnosing, prognosing, or determining a likelihood of developing of a disease or condition associated with or arising from reduced insulin production in the subject.

The ten or more extracellular microRNAs may be any combination of the listed microRNAs, provided that the combination comprises ten or more of the microRNAs.

### Embodiment 49

Use of one or more agents for determining expression levels of ten or more extracellular microRNAs selected from the group consisting of: miR-24, miR-26a, miR-146a, mmiR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from a subject, for the preparation of a medicament for monitoring the response of the subject to a treatment for a disease or condition associated with reduced insulin production.

The ten or more extracellular microRNAs may be any combination of the listed microRNAs, provided that the combination comprises ten or more of the microRNAs.

### Embodiment 50

The use of embodiment 49, wherein the treatment comprises beta-islet cell transplantation, and/or the administration of a vaccine or therapeutic drug designed to retard loss of beta-islet cells in the subject and/or loss of insulin production capacity in beta-islet cells of the subject.

### Embodiment 51

Use of one or more agents for determining expression levels of ten or more extracellular microRNAs selected from the group consisting of: miR-24, miR-26a, miR-146a, mmiR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from a subject, for the preparation of a medicament for assessing the efficacy of a treatment for inhibiting or preventing loss of beta-islet cell insulin production function and/or beta-islet cell death in the subject.

The ten or more extracellular microRNAs may be any combination of the listed microRNAs, provided that the combination comprises ten or more of the microRNAs.

### Embodiment 52

The use of any one of embodiments 48 to 51, wherein the microRNAs comprise or consist of any: 10, 11, 12, 13, 14, 15, 16, 17, or 18; of said microRNAs.

### Embodiment 53

The use of any one of embodiments 48 to 52, wherein the subject is of Australian ethnicity, of Chinese ethnicity, or of Indian ethnicity.

### Embodiment 54

The use of any one of embodiments 48 to 53, wherein the microRNAs are selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

### Embodiment 55

The use of embodiment 54, wherein the microRNAs comprise or consist of any: 10, 11, 12, 13, 14, 15, 16, 17, 18, 19, 20, 21, 22, or 23; of said microRNAs.

### Embodiment 56

The use of embodiment 54 or embodiment 55, wherein the subject is of Australian ethnicity or of Chinese ethnicity.

### Embodiment 57

The use of any one of embodiments 48 to 53, wherein the ten or more microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-146a, miR-186, miR-199a-3p, miR-222, miR-223, miR-340 and miR-27a.

### Embodiment 58

The use of embodiment 57, wherein the microRNAs comprise or consist of any: 10, 11, 12, 13, 14, 15, 16, 17, 18, 19, or 20; of said microRNAs.

### Embodiment 59

The use of embodiment 57 or embodiment 58, wherein the subject is of Australian ethnicity or of Indian ethnicity.

### Embodiment 60

The use of of any one of embodiment 48 to 53, wherein the ten or more microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

### Embodiment 61

The use of embodiment 60, wherein the microRNAs comprise or consist of any: 10, 11, 12, 13, 14, 15, 16, 17, 18, or 19; of said microRNAs.

### Embodiment 62

The use of embodiment 60 or embodiment 61, wherein the subject is of Chinese ethnicity or of Indian ethnicity.

### Embodiment 63

The use of any one of embodiments 48 to 53, wherein the ten or more microRNAs are selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223

### Embodiment 64

The use of embodiment 63, wherein the microRNAs comprise or consist of any: 10, 11, 12, 13, 14, 15, 16, 17, 18, 19, 20, 21, 22, 23, 24, 25, 26, 27, 28 or 29; of said microRNAs.

### Embodiment 65

The use of embodiment 63 or embodiment 64, wherein the subject is of Australian ethnicity.

### Embodiment 66

The use of any one of embodiments 48 to 53, wherein the ten or more microRNAs are selected from the group consisting of: let-7e, miR-7, miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

### Embodiment 67

The use of embodiment 66, wherein the microRNAs comprise or consist of any: 10, 11, 12, 13, 14, 15, 16, 17, 18, 19, 20, 21, 22, 23, 24, 25, 26, 27, 28 or 29; of said microRNAs.

### Embodiment 68

The use of embodiment 66 or embodiment 67, wherein the subject is of Chinese ethnicity.

The present invention also relates at least in part to the following embodiments:

### Embodiment 1

A method for diagnosing, prognosing, or determining a likelihood of developing a disease or condition associated with or arising from reduced insulin production in a subject, the method comprising:

determining expression levels of one or more extracellular microRNA/s selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from the subject; and

comparing expression levels the microRNA/s in the biological fluid sample to control expression level/s of the microRNA/s generated from control biological fluid/s equivalent to the sample,

wherein elevated expression levels of the microRNA/s in the biological fluid sample compared to the expression level/s of the microRNA/s generated from the control biological fluid/s is indicative of a deficient or absent capacity for insulin production in beta-islet cells of the subject, and

the reduced or absent insulin production in the beta-islet cells is:

(i) predictive that the subject will develop the disease or condition; or

(ii) diagnostic of the disease or condition in the subject; or

(iii) prognostic that the disease or condition will progress in the subject.

The one or more microRNAs include any combination of the listed microRNAs, provided that the combination comprises two or more of the microRNAs.

### Embodiment 2

A method for monitoring the response of a subject to a treatment for a disease or condition associated with or arising from reduced insulin production in a subject, the method comprising:

determining expression levels of one or more extracellular microRNA/s selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from the subject; and

comparing expression levels the microRNA/s in the biological fluid sample to control expression level/s of the microRNA/s generated from control biological fluid/s equivalent to the sample,

wherein elevated expression levels of the microRNA/s in the biological fluid sample compared to the expression level/s of the microRNA/s generated from the control biological fluid/s is indicative of deficient or absent capacity for insulin production in beta islet cells of the subject, and a deficient or absent response to the treatment by the subject.

The one or more microRNAs include any combination of the listed microRNAs, provided that the combination comprises two or more of the microRNAs.

### Embodiment 3

The method of embodiment 2, wherein the treatment comprises beta-islet cell transplantation, and/or the administration of a vaccine or therapeutic drug designed to retard loss of beta-islet cells in the subject and/or loss of insulin production capacity in beta-islet cells of the subject.

### Embodiment 4

A method for assessing the efficacy of a treatment for inhibiting or preventing loss of beta-islet cell insulin production function and/or beta-islet cell death, the method comprising:

administering the treatment to a subject in need thereof,

determining expression levels of one or more extracellular microRNA/s in a biological fluid sample obtained from the subject after said administering of the treatment, wherein the microRNA/s are selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; and

comparing expression levels the microRNA/s in the biological fluid sample to control expression level/s of the microRNA/s generated from control biological fluid/s equivalent to the sample,

wherein equivalent or reduced expression levels of the microRNA/s in the biological fluid sample compared to the expression level/s of the microRNA/s generated from the control biological fluid/s is indicative that the treatment has efficacy in inhibiting or preventing loss of beta-islet cell insulin production function and/or beta-islet cell death.

The one or more microRNAs include any combination of the listed microRNAs, provided that the combination comprises two or more of the microRNAs.

### Embodiment 5

The method of any one of embodiments 1 to 4, further comprising removing any cells comprising nuclear material present in the biological fluid prior to determining expression levels of one or more extracellular microRNA/s.

### Embodiment 6

The method of embodiment 5, wherein said removing of the cells comprising nuclear material comprises lysing less than: 10%, 5%, 4%, 3%, 2%, 1%, 0.5%, 0.2% or 0.1% of the cells.

### Embodiment 7

The method of any one of embodiments 1 to 6, wherein the condition or disease is selected from any of diabetes, pancreatitis, insulinoma, and pancreatic cancer.

### Embodiment 8

The method of embodiment 7, wherein the disease is diabetes.

### Embodiment 9

The method of embodiment 8, wherein the diabetes is Type 1 diabetes.

### Embodiment 10

The method of any one of embodiments 1 to 9, wherein the biological fluid sample is whole blood.

### Embodiment 11

The method of any one of embodiments 1 to 9, wherein the biological fluid sample is serum or plasma.

### Embodiment 12

The method of any one of embodiments 1 to 11, further comprising an initial step of obtaining the biological fluid sample from the subject.

### Embodiment 13

The method of any one of embodiments 1 to 12, wherein the microRNA/s comprise or consist of any: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51; of said microRNAs.

### Embodiment 14

The method of any one of embodiments 1 to 13, wherein the microRNA/s are selected from the group consisting of: miR-125b, miR-127, miR-125a-5p, miR-24, miR-29a, miR-27b, miR-99b, miR-181a, miR-223, miR-222, miR-26a, miR-375, miR-30c, miR-374, miR-30b, miR-15b, miR-103, let-7e, miR-7, miR-92a, miR-93, miR-146a, miR-16, miR-200a, miR-26b, miR-186, miR-25, miR-30a-5p, miR-20a, miR-145, miR-199a-3p, miR-148a, miR-30e-3p, miR-22-5p, miR-21, miR-27a, snRNA-U6, miR-152 and miR-34a.

### Embodiment 15

The method of embodiment 14, wherein the microRNA/s comprise or consist of any: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39; of said microRNAs.

### Embodiment 16

The method of any one of embodiments 1 to 13, wherein the one or more microRNA/s comprise or consist of: miR-409-5p, miR-340#, miR-301b, miR-155, miR-220c, miR-558, miR-625, miR-9, miR-188, miR-126, miR-210 and/or miR-326.

### Embodiment 17

The method of embodiment 16, wherein the microRNA/s comprise or consist of any: 2, 3, 4, 5, 6, 7, 8, 9, 10, 11, or 12; of said microRNAs.

### Embodiment 18

The method of any one of embodiments 1 to 13, wherein the one or more microRNA/s comprise or consist of: miR-125b, miR-127, miR-125a-5p, miR-24, miR-29a, miR-27b and/or miR-99b.

### Embodiment 19

The method of embodiment 18, wherein the microRNA/s comprise or consist of any: 2, 3, 4, 5, 6, or 7 of said microRNAs.

### Embodiment 20

A kit comprising primers, probes and/or other binding agents for use in detecting expression of at least two microRNAs selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from the subject.

The at least two microRNAs include any combination of the listed microRNAs, provided that the combination comprises two or more of the microRNAs.

### Embodiment 21

A microRNA signature comprising at least two microRNAs selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from the subject.

The at least two microRNAs include any combination of the listed microRNAs, provided that the combination comprises two or more of the microRNAs.

### Embodiment 22

The kit of embodiment 20 or the microRNA signature of embodiment 21, wherein the microRNA/s comprise or consist of any: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51; of said microRNAs.

### Embodiment 23

The kit of embodiment 20 or embodiment 22, or the microRNA signature of embodiment 19 or embodiment 20, wherein the microRNA/s are selected from the group consisting of: miR-125b, miR-127, miR-125a-5p, miR-24, miR-29a, miR-27b, miR-99b, miR-181a, miR-223, miR-222, miR-26a, miR-375, miR-30c, miR-374, miR-30b, miR-15b, miR-103, let-7e, miR-7, miR-92a, miR-93, miR-146a, miR-16, miR-200a, miR-26b, miR-186, miR-25, miR-30a-5p, miR-20a, miR-145, miR-199a-3p, miR-148a, miR-30e-3p, miR-22-5p, miR-21, miR-27a, snRNA-U6, miR-152 and miR-34a.

### Embodiment 24

The kit or microRNA signature of embodiment 23, wherein the microRNA/s comprise or consist of any: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39; of said microRNAs.

### Embodiment 25

The kit of embodiment 20 or embodiment 22, or the microRNA signature of embodiment 21 or embodiment 22, wherein the one or more microRNA/s comprise or consist of: miR-409-5p, miR-340#, miR-301b, miR-155, miR-220c, miR-558, miR-625, miR-9, miR-188, miR-126, miR-210 and/or miR-326.

### Embodiment 26

The kit or microRNA signature of embodiment 25, wherein the microRNA/s comprise or consist of any: 2, 3, 4, 5, 6, 7, 8, 9, 10, 11, or 12; of said microRNAs.

### Embodiment 27

The kit of embodiment 20 or embodiment 22, or the microRNA signature of embodiment 21 or embodiment 22, wherein the one or more microRNA/s comprise or consist of: miR-125b, miR-127, miR-125a-5p, miR-24, miR-29a, miR-27b and/or miR-99b.

### Embodiment 28

The kit or microRNA signature of embodiment 27, wherein the microRNA/s comprise or consist of any: 2, 3, 4, 5, 6, or 7; of said microRNAs.

### Embodiment 29

The kit of any one of embodiments 20 or 22 to 28, further comprising any one or more of:

(i) one or more reagents for extracting the biological fluid sample;

(ii) one or more reagents for reverse-transcribing RNA into cDNA;

(iii) one or more reagents for amplifying the cDNA;

(iv) control standards for normalisation of microRNA signature expression data and/or comparison of microRNA signature expression data to determine whether expression of the microRNA signature is increased, reduced, and/or within a standard range;

(v) any one of more of: buffers, washing reagents, RNAse inhibitors, precipitators (e.g. glycogen), nuclease-free water, salt solutions.

### Embodiment 30

Use of the kit of any one of embodiments 20 or 22 to 29, or the microRNA signature of any one of embodiments embodiment 21 to 28, for diagnosing, prognosing, or determining a likelihood of developing a disease or condition associated to with or arising from reduced insulin production in a subject, wherein the disease or condition is selected from the group consisting of diabetes, pancreatitis, insulinoma, and pancreatic cancer.

### Embodiment 31

Use of one or more agents for determining expression levels of one or more extracellular microRNA/s selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from a subject, for the preparation of a medicament for diagnosing, prognosing, or determining a likelihood of developing of a disease or condition associated with or arising from reduced insulin production in the subject.

The one or more microRNAs include any combination of the listed microRNAs, provided that the combination comprises two or more of the microRNAs.

### Embodiment 32

Use of one or more agents for determining expression levels of one or more extracellular microRNA/s selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from a subject, for the preparation of a medicament for monitoring the response of the subject to a treatment for a disease or condition associated with reduced insulin production.

### Embodiment 33

The use of embodiment 32, wherein the treatment comprises beta-islet cell transplantation, and/or the administration of a vaccine or therapeutic drug designed to retard loss of beta-islet cells in the subject and/or loss of insulin production capacity in beta-islet cells of the subject.

### Embodiment 34

Use of one or more agents for determining expression levels of one or more extracellular microRNA/s selected from the group consisting of: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326; in a biological fluid sample obtained from a subject, for the preparation of a medicament for assessing the efficacy of a treatment for inhibiting or preventing loss of beta-islet cell insulin production function and/or beta-islet cell death in the subject.

### Embodiment 35

The use of any one of embodiments 31 to 34, wherein the microRNA/s comprise or consist of any: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51; of said microRNAs.

### Embodiment 36

The use of any one of embodiments 31 to 35, wherein the microRNA/s are selected from the group consisting of: miR-125b, miR-127, miR-125a-5p, miR-24, miR-29a, miR-27b, miR-99b, miR-181a, miR-223, miR-222, miR-26a, miR-375, miR-30c, miR-374, miR-30b, miR-15b, miR-103, let-7e, miR-7, miR-92a, miR-93, miR-146a, miR-16, miR-200a, miR-26b, miR-186, miR-25, miR-30a-5p, miR-20a, miR-145, miR-199a-3p, miR-148a, miR-30e-3p, miR-22-5p, miR-21, miR-27a, snRNA-U6, miR-152 and miR-34a.

### Embodiment 37

The use of embodiment 36, wherein the microRNA/s comprise or consist of any: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39; of said microRNAs.

### Embodiment 38

The use of any one of embodiments 31 to 35, wherein the one or more microRNA/s comprise or consist of: miR-409-5p, miR-340#, miR-301b, miR-155, miR-220c, miR-558, miR-625, miR-9, miR-188, miR-126, miR-210 and/or miR-326.

### Embodiment 39

The use of embodiment 38, wherein the microRNA/s comprise or consist of any: 2, 3, 4, 5, 6, 7, 8, 9, 10, 11, or 12; of said microRNAs.

### Embodiment 40

The use of any one of embodiments 31 to 35, wherein the one or more microRNA/s comprise or consist of: miR-125b, miR-127, miR-125a-5p, miR-24, miR-29a, miR-27b and/or miR-99b.

### Embodiment 41

The use of embodiment 40, wherein the microRNA/s comprise or consist of any: 2, 3, 4, 5, 6, or 7 of said microRNAs.

### Embodiment 42

The method of any one of embodiments 1 to 19, wherein the control biological fluid/s is/are obtained from control subject/s determined not to have a disease or condition associated with aberrant insulin production and/or abnormal insulin metabolism.

### Embodiment 43

The method of embodiment 42, wherein the control subject/s are a population of individuals determined not to have a disease or condition associated with aberrant insulin production and/or abnormal insulin metabolism.

### Embodiment 44

The method of embodiment 42 or embodiment 43, wherein the control subject/s do not have a disease or condition selected from the group consisting of diabetes, pancreatitis, insulinoma, and pancreatic cancer.

### Definitions

As used in this application, the singular form “a”, “an” and “the” include plural references unless the context clearly dictates otherwise. For example, the term “microRNA” also includes a plurality of microRNAs.

As used herein, the term “comprising” means “including.” Variations of the word “comprising”, such as “comprise” and “comprises,” have correspondingly varied meanings. Thus, for example, a composition “comprising” microRNA type A may consist exclusively of microRNA type A or may include one or more additional components (e.g. microRNA type B).

As used herein, a disease or condition that is “associated with aberrant insulin production” will be understood to encompass any ailment that arises directly and/or indirectly from aberrant insulin production, and/or that causes aberrant insulin production in a subject, “aberrant insulin production” meaning levels of insulin production that lie outside (e.g. reduced or increased) of a standard physiological range of a population of individuals (e.g. a multi-ethnic population) of the same species as the subject determined to have non-aberrant (i.e. normal, standard) insulin production. The population may also be of the same or similar: race, gender, sex, and/or age as the subject. The determination of aberrant insulin production in a given subject may be achieved using standard tests known in the art.

As used herein, a disease or condition that is “associated with or arising from reduced insulin production” will be understood to encompass any ailment that arises directly and/or indirectly from a reduction in insulin production in a subject (e.g. a reduction in the production of insulin by insulin-producing cells in the subject, including but not limited to pancreatic cells (including beta-islet cells), brain cells, and/or gall bladder cells). Non-limiting examples of such diseases or conditions include diabetes (e.g. Type 1, Type 2), pancreatitis, insulinoma, and some forms of pancreatic cancer).

As used herein, a “cell-free” or “circulating” microRNA or microRNA signature will be understood to mean microRNA(s) located externally of cells within a subject, rather than intracellularly.

As used herein, the term “subject” includes any animal of economic, social or research importance including bovine, equine, ovine, primate, avian and rodent species. Hence, a “subject” may be a mammal such as, for example, a human or a non-human mammal.

As used herein, the term “kit” refers to any delivery system for delivering materials. Such delivery systems include systems that allow for the storage, transport, or delivery of reaction reagents (for example labels, reference samples, supporting material, etc. in the appropriate containers) and/or supporting materials (for example, buffers, written instructions for performing an assay etc.) from one location to another. For example, kits may include one or more enclosures, such as boxes, containing the relevant reaction reagents and/or supporting materials. The term “kit” includes both fragmented and combined kits. A “fragmented kit” refers to a delivery system comprising two or more separate containers that each contains a sub-portion of the total kit components. The containers may be delivered to the intended recipient together or separately. Any delivery system comprising two or more separate containers that each contains a sub-portion of the total kit components are included within the meaning of the term “fragmented kit”. A “combined kit” refers to a delivery system containing all of the components of a reaction assay in a single container (e.g. in a single box housing each of the desired components).

It will be understood that use the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a polypeptide of between 10 residues and 20 residues in length is inclusive of a polypeptide of 10 residues in length and a polypeptide of 20 residues in length.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### BRIEF DESCRIPTION OF THE FIGURES

Preferred embodiments of the present invention will now be described by way of example only, with reference to the accompanying figures wherein:

Figure One provides a series of graphs showing microRNA levels in pancreatic islet cell supernatant.

Figure Two provides a graph (A) and a series of microscopy images (B) and (C) parts i)-vii) showing insulitis levels in non-obese diabetic (NOD) mice.

Figure Three provides a series of graphs demonstrating that the abundance of some circulating microRNAs changes over time in NOD mice.

Figure Four provides a series of graphs demonstrating that the abundance of some circulating microRNAs remains stable over time in NOD mice.

Figure Five shows the correlation between 18 microRNAs and fasting blood glucose levels (BGLs) in NOD mice (A). The graphs in (B) parts (i)-(iii) are three representative microRNAs plotted against the respective fasting BGL using Spearman ranks. Data from 16 and 18-week old mice were omitted. Colours correspond to highlighted microRNAs in (A).

Figure Six relates to the profiling of circulating microRNA in subjects with type 1 diabetes (T1D). (A) volcano plot of differentially abundant microRNAs measured within the circulation of patients with newly-diagnosed (ND-T1D) and established (E-T1D) T1D, compared to age and gender-matched controls (Con(ND) and Con(E) respectively). (B) parts (i)-(iii) are graphs depicting a subset of significant microRNAs highlighted in (A) with newly diagnosed T1D and controls in green and established T1D and controls in blue.

Figure Seven provides two graphs (A) and (B) showing transcript abundance of microRNAs found to be significantly elevated in the circulation of individuals with established T1D compared to age and gender-matched controls.

Figure Eight provides a series of graphs (A)-(E) showing transcript abundance of microRNAs found to be significantly elevated in the circulation of T1D patients with detectable C-peptide (C-pep+), compared to those without detectable C-peptide (C-pep−).

Figure Nine shows a heatmap of microRNA signature expression in high risk and T1D individuals.

Figure Ten provides a series of graphs evidencing that certain microRNAs are elevated in the circulation of individuals at high risk for T1D, and at T1D diagnosis.

Figure Eleven provides two graphs evidencing two specific microRNAs elevated in the circulation of individuals at T1D diagnosis.

Figure Twelve is a Venn diagram of PREDICT T1D microRNAs found to be elevated in NOD mice (blue circle), released after human islet SNP exposure (yellow circle), elevated in individuals at high risk of T1D or at the point of diagnosis (red circle), or a combination of all three. MicroRNA-9, -126, -155, -188, -210, -220c, -301b, -326, -340-3p, -409-5p, -558, and -625 were not found to significantly change in any of these studies.

Figure Thirteen is a graph showing the effect of ingested interferon-α on residual C-peptide at one year following treatment with 5000 units, 30,000 units or placebo. Treatment groups are shown on the X-axis. The remaining beta-cell function measured as circulating C-peptide levels at 1 year (relative to baseline) are plotted on Y-axis (adjusted means, vertical bars denote 0.95 confidence intervals).

Figure Fourteen provides a series of graphs demonstrating that the microRNA signature of beta cell death decreased in individuals showing the highest preservation of beta-cell mass at one year of interferon-α treatment.

Figure Fifteen is a series of graphs depicting the results of a univariate analysis of the 50 microRNA panel on samples from Australian subjects (type-I diabetes/“T1D” and healthy controls/“Control”).

Figure Sixteen is a series of graphs depicting the results of a univariate analysis of the 50 microRNA panel on samples from Indian subjects (type-1 diabetes/“T1D” and healthy control s/“Control”).

Figure Seventeen is a series of graphs depicting the results of a univariate analysis of the 50 microRNA panel on samples from Chinese subjects (type-1 diabetes/“T1D” and healthy controls/“Control”).

Figure Eighteen shows the results of a random forest analysis of the 50 microRNA panel on samples from Australian subjects (type-I diabetes and healthy controls).

Figure Nineteen shows the results of a random forest analysis of the 50 microRNA panel on samples from Indian subjects (type-1 diabetes and healthy controls).

Figure Twenty shows the results of a random forest analysis of the 50 microRNA panel on samples from Chinese subjects (type-1 diabetes and healthy controls).

Figure Twenty-one shows the results of a ROC analysis of samples from Indian subjects using a model derived from the 31 most important miRNAs (miRNAs with green-labeling in Figure eighteen) panel derived from the random forest analysis on samples from Australian subjects.

Figure Twenty-two shows the results of a ROC analysis of samples from Chinese subjects using the 31 most important miRNAs (miRNAs with green-labeling in Figure eighteen) panel derived from the random forest analysis on samples from Australian subjects.

Figure Twenty-three is a Venn diagram summarising the most important microRNAs (microRNAs with green-labeling in Figure Eighteen, Figure Nineteen and Figure Twenty) resulting from the random forest analysis of the 50 microRNA panel data on samples from Australian, Indian and Hong Kong Chinese subjects (type-1 diabetes Vs healthy controls).

### DETAILED DESCRIPTION

The present invention relates to circulating microRNA signatures indicative of, for example, beta-cell death. Without limitation, the microRNA signatures described herein may be used: (i) as biomarkers to inform for predicting, diagnosing, and/or prognosing the development of diseases and conditions associated with aberrant (e.g. reduced) insulin production (e.g. Type I diabetes); (ii) to monitor responses to interventions such as islet transplantation, vaccines and drugs aiming to retard β-cell loss; (iii) to select treatments to block β-cell death; and/or (iv) to guide the development of new treatments to lessen the burden of development diseases and conditions associated with aberrant (e.g. reduced) insulin production.

The microRNA signatures of the present invention and non-limiting examples of their applications are described in detail as follows.

microRNA Signatures

The present invention provides cell-free microRNA signatures indicative of beta-cell insulin production capacity.

The cell-free microRNA signatures may comprise or consist of any one or more of the following microRNAs, in any combination: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326. For example, the microRNA signatures may comprise or consist of any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 of these microRNAs.

In some embodiments the cell-free microRNA signatures may comprise or consist of any one or more of the following microRNA/s, in any combination: miR-125b, miR-127, miR-125a-5p, miR-24, miR-29a, miR-27b, miR-99b, miR-181a, miR-223, miR-222, miR-26a, miR-375, miR-30c, miR-374, miR-30b, miR-15b, miR-103, let-7e, miR-7, miR-92a, miR-93, miR-146a, miR-16, miR-200a, miR-26b, miR-186, miR-25, miR-30a-5p, miR-20a, miR-145, miR-199a-3p, miR-148a, miR-30e-3p, miR-22-5p, miR-21, miR-27a, snRNA-U6, miR-152 and miR-34a. For example, the microRNA signatures may comprise or consist of any 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of these microRNAs.

In still other embodiments, the cell-free microRNA signatures may comprise or consist of miR-21, miR-223, miR-24, miR-26a, miR-29a, and miR-326.

In other embodiments, the cell-free microRNA signatures may comprise or consist of miR-125b, miR-127, miR-125a-5p, miR-24, miR-29a, miR-27b and miR-99b.

In other embodiments, the cell-free microRNA signatures may comprise or consist of miR-409-5p, miR-340#, miR-301b, miR-155, miR-220c, miR-558, miR-625, miR-9, miR-188, miR-126, miR-210 and/or miR-326.

In still other embodiments, the cell-free microRNA signatures may comprise or consist of five or more, six or more, seven or more, eight or more, nine or more, or ten or more, extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93.

In still other embodiments, the cell-free microRNA signatures may comprise or consist of five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, or twenty-three or more, microRNAs selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

In still other embodiments, the cell-free microRNA signatures may comprise or consist of five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more, microRNAs selected from the group consisting of: miR-16, miR-20a, miR-21, miR-24, miR-25, miR-26a, miR-26b, miR-30a-5p, miR-30b, miR-30c, miR-92a, miR-93, miR-126, miR-146a, miR-186, miR-199a-3p, miR-222, miR-223, miR-340 and miR-27a.

In still other embodiments, the cell-free microRNA signatures may comprise or consist of five or more, six or more, seven or more, eight or more, nine or more, ten or more, eleven or more, twelve or more, thirteen or more, fourteen or more, fifteen or more, sixteen or more, seventeen or more, eighteen or more, or nineteen or more, microRNAs selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

In still other embodiments, the cell-free microRNA signatures may comprise or consist of five or more, six or more, seven or more, eight or more, nine or more, ten or more, eleven or more, twelve or more, thirteen or more, fourteen or more, fifteen or more, sixteen or more, seventeen or more, eighteen or more, or nineteen or more, twenty or more, twenty-one or more, twenty-two or more, twenty-three or more, twenty-four or more, twenty-five or more, twenty-six or more, twenty-seven or more, twenty-eight or more, or twenty-nine or more, microRNAs selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223.

In still other embodiments, the cell-free microRNA signatures may comprise or consist of five or more, six or more, seven or more, eight or more, nine or more, ten or more, eleven or more, twelve or more, thirteen or more, fourteen or more, fifteen or more, sixteen or more, seventeen or more, eighteen or more, or nineteen or more, twenty or more, twenty-one or more, twenty-two or more, twenty-three or more, twenty-four or more, twenty-five or more, twenty-six or more, twenty-seven or more, twenty-eight or more, or twenty-nine or more, microRNAs selected from the group consisting of: 26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

Detecting microRNA Signatures

The microRNA signatures of the present invention can be detected in a biological sample using standard methods known in the art.

Methods of RNA extraction suitable for use in generating microRNA signatures of the present invention are well known in the art. Without limitation, suitable methods are disclosed in the Examples of the present application, as well as standard textbooks including Ausubel et al., Ed., “Current Protocols in Molecular Biology”, John Wiley & Sons, New York 1987-1999. RNA isolation may be performed using commercially available purification kits, buffer sets and proteases according to the manufacturer's recommended instructions (see for example, commercial kits available from Thermo Fisher Scientific, Sigma-Aldrich, Roche, Promega and Qiagen). RNA can be isolated from blood or purified components thereof (e.g. serum, plasma) using standard methods known in the art. Commercially-available kits for this purpose include those provided by ThermoFisher Scientific (TRIzol® LS PureLink™ Total RNA Blood Kit MagMAX™ for Stabilized Blood Tubes RNA Isolation Kit MagMAX mirVana Total RNA Isolation RiboPure™ Blood Kit), Qiagen (QIAamp RNA Blood Mini Kit, Qiagen Circulating Nucleic Acid Kit, Qiagen miRNEasy, QiaSymphony RNA extraction kit), ThermoFisher Scientific Ambion TRIzol LS Reagent, Exiqon MiRCURY RNA Isolation Kit, and Promega (Maxwell® CSC Blood RNA Kit).

Expression levels of specific microRNAs that in combination make up the microRNA signatures of the present invention can be determined using conventional methods known in the art (e.g. polymerase-based assays, hybridisation-based assays, flap endonuclease-based assays, direct RNA capture with branched DNA, and the like). Non-limiting methods suitable for detecting the level of expression of a given microRNA in a biological sample include microarray profiling, RT-PCR, Northern blotting, differential display, reporter gene matrix assays, nuclease protection, slot or dot blots, ICAT, 2D gel electrophoresis, SELDI-TOF, assays using MNAzymes/PlexZymes, enzyme assays, and antibody assays. Although not required, microRNAs under analysis for expression may be amplified using known techniques including, for example, any one or more of: the polymerase chain reaction (PCR), reverse transcription-polymerase chain reaction (RT-PCR), nucleic acid sequence-based amplification (NASBA), loop-mediated isothermal amplification (LAMP), self-sustained sequence replication (3SR), rolling circle amplification (RCA), transcription-mediated amplification (TMA), and strand displacement amplification (SDA).

Suitable high throughput methods suitable for microRNA quantification may include those involving physical or logical arrays.

Non-limiting examples include assays which utilise solid phase arrays. Exemplary formats include membrane or filter arrays (e.g. nylon, nitrocellulose), bead arrays, and pin arrays. In general, the solid phase assays may utilise probes that specifically interact with (e.g. bind or hybridise to) a microRNA expression product may be immobilised, to a solid support (e.g. by indirect or direct cross-linking). Any solid support compatible with assay reagents and conditions may be utilised (e.g. silicon, modified silicon, silicon dioxide, various polymers (e.g. polystyrene, polycarbonate, (poly)tetrafluoroethylene, (poly)vinylidenedifluoride, or combinations thereof) or functionalised glass). In some embodiments, the solid support may be a chip composed wholly or partially of any one or more of silicon, modified silicon, silicon dioxide, various polymers (e.g. polystyrene, polycarbonate, (poly)tetrafluoroethylene, (poly)vinylidenedifluoride, or combinations thereof) or functionalised glass). Binding proteins (e.g. antibodies, antigen-binding fragments, or derivatives thereof) or polynucleotide probes, (e.g. DNA, RNA, cDNA, synthetic oligonucleotides, and the like) which specifically interact with target microRNA/s may be immobilised on the chip in an array (i.e. a logically-ordered manner) for detection of any microRNAs in a sample applied thereto.

Microarray expression may be detected by scanning the microarray using any of a variety of CCD-based or laser scanners, and analysing output using any suitable software, (e.g. GENEPIX™ (Axon Instruments), nCounter® (NanoString Technologies), IMAGENE™ (Biodiscovery), Feature Extraction Software (Agilent)).

Non-limiting examples include assays which utilise liquid phase arrays (e.g. for hybridisation of nucleic acids, binding of antibodies or other receptors to a ligand) in microtiter or multiwell plates. Non-limiting examples of suitable systems include xMAP® (Luminex), ORCA™ (Beckman-Coulter, Inc.) SECTOR® Imager with MULTI-ARRAY® and MULTI-SPOT systems (Meso Scale Discovery), miRCURY LNA™ microRNA Arrays (Exiqon), and ZYMATE™ (Zymark Corporation).

Reverse transcription PCR and real-time PCR may be employed to determine levels of microRNA expression in accordance with the invention. Two commonly used quantitative RT-PCR techniques are the Lightcycler assay (Roche, USA) and the TaqMan RT-PCR assay (ABI, Foster City, USA). Commercial RT-PCR products for assessing microRNA levels include the TaqMan Low-Density miRNA Array card (Applied Biosystems). Art-known methods of expression profiling of microRNAs using real-time quantitative PCR are described, for example, in Chen et al. (2009), BMC Genomics, 10:407, and Benes and Castaldi (2010), Methods, 50:244-249.

Data indicative of microRNA expression levels may be normalised against the expression level of a suitable control RNA. The normalised data may then be processed using appropriate software to generate a microRNA signature (e.g. represented by a numeric number) representative of the expression level profile of the microRNAs. This signature may be compared with a reference value to assess whether it is indicative of a low expression or a high expression of the microRNAs in question. The reference value can be determined based on microRNA signatures (including the same microRNA signature) obtained from control patient/s (e.g. those with non-aberrant insulin production) via computational analysis. For example, the reference value may be the middle point between the signature of subject/s determined to have aberrant insulin production and subject/s determined to have non-aberrant insulin production.

Various computer software may be utilised in determining a microRNA signature of the present invention. Non-limiting examples include Plausible Neural Network (PNN) (see, for example, U.S. Pat. No. 7,287,014), PNN Solution software (PNN Technologies Inc.), Prediction Analysis of Microarray (PAM) (see, for example, Tibshirani et al. (2002), PNAS 99(10):6567-6572), and Significance Analysis of Microarray (SAM).

The skilled person will recognise that methods disclosed above are exemplary and any suitable method of determining microRNA expression may be utilised.

### Methods for Predicting, Diagnosis, and Prognosis

The cell-free (i.e. circulating) microRNA signatures of the present invention may be used to detect the loss of insulin-producing beta-cells, and can thus be used as a measure of insulin-production capacity in a test sample.

Given the central role of reduced beta-cell insulin-production in various diseases and conditions, the microRNA signatures disclosed herein may be used as biomarkers to inform for predicting, diagnosing, and/or prognosing the development of diseases and conditions associated with reduced insulin production. Accordingly, the microRNA signatures described herein can be used, for example, to identify and/or monitor a subject suspected to be at risk of developing a disease or condition associated with reduced insulin production. Alternatively, they may be used to diagnose a subject with a disease or condition associated with reduced insulin production. Alternatively, the microRNA signatures may be used to predict the progression of the disease or condition associated with or arising from reduced insulin production in a subject.

Without any particular limitation, the subject may be a mammal such as, for example, a human or a non-human mammal. The human subject may, in some embodiments, be of a particular ethnicity including, for example, Caucasian, Asian, African, Latino, Hispanic, European, pacific islander, white, or black.

Non-limiting examples of white ethnic subjects include those of European, Australian, and North American origin.

Non-limiting examples of Indian ethnic subjects include those of the Indian subcontinent.

Non-limiting examples of Asian ethnic subjects include those of the Far East, and Southeast Asia, including, for example, Cambodia, China, Japan, Korea, Malaysia, Pakistan, the Philippine Islands, Thailand and Vietnam.

Non-limiting examples of Hispanic or Latino ethnic subjects include those of Cuban, Mexican, Puerto Rican, South or Central American or other Spanish culture origin.

Non-limiting examples of pacific islander ethnic subjects include those of Native Hawaiian, Guamian or Chamorro, Samoan, and other Pacific Islander origin.

Without any particular limitation, the microRNA signatures may be used to predict, diagnose, and/or prognose the development of diseases and conditions including metabolic diseases related to glucose-insulin metabolism or cancer related to endocrine cells. In some embodiments, the disease is Type 1 diabetes.

The microRNA signatures described herein may be used to monitor the response of a subject to a treatment administered for the purpose of alleviating, curing, and/or reducing the symptoms associated with a disease or condition associated with reduced insulin production. For example and without limitation, a determination that the subject is undergoing an increased expression of a given microRNA signature described herein in response to a given treatment or therapeutic intervention may be indicative of a negative or absent response to the treatment or therapeutic intervention by the subject. Alternatively, a determination that the subject does not have an increased expression, or has a reduced expression, of a given microRNA signature described herein in response to a given treatment or therapeutic intervention may be indicative of a positive response to the treatment or therapeutic intervention by the subject. Without any particular limitation, the microRNA signatures may be used to monitor the response of the subject to treatments and therapeutic interventions for diseases and conditions including any form/type of diabetes, pancreatitis, insulinoma, other common or rare forms of pancreatic cancers, and any treatments/therapies related to these. In some embodiments, the disease is Type 1 diabetes. The treatment or therapeutic intervention may comprise any one or more of administering pharmaceutical agents (e.g. vaccines, drugs, therapeutic agents, nanoparticles) to the subject, the grafting of cells (beta-islet cell transplantation), and/or the use of insulin (or dual/multi-hormone) replacement devices.

Additionally or alternatively, the microRNA signatures described herein may be used to identify and/or test the efficacy of a treatment or therapeutic intervention. For example and without limitation, a determination that the subject is undergoing an increased expression of a given microRNA signature described herein in response to a given candidate treatment or therapeutic intervention may be indicative that the treatment or therapeutic intervention is ineffective against the targeted disease or condition associated with reduced insulin production. Alternatively, a determination that the subject does not have an increased expression, or has a reduced expression, of a given microRNA signature described herein in response to a given candidate treatment or therapeutic intervention may be indicative that the treatment or therapeutic intervention is effective against the targeted disease or condition associated with reduced insulin production. Without any particular limitation, the targeted disease or condition may include any form/type of diabetes, pancreatitis, insulinoma, other common or rare forms of pancreatic cancers, and any form or type of treatments related to these. In some embodiments, the disease is Type 1 diabetes.

In general, detection of an increased expression of a microRNA signature as described herein is indicative of loss of beta-islet cell number and or function, and a consequent indication of reduced insulin production capacity in the subject. Alternatively, detection of a reduced expression of a microRNA signature as described herein in a test subject is indicative that beta-islet cell number and or function is not compromised in the subject. Determination of whether expression of a given microRNA signature is increased or reduced is generally made by comparison of the subject expression levels to the expression levels of the same microRNA signature (or expression levels of individual microRNAs within the signature) obtained from a control subject, or a population of control subjects.

The control subject population may be of the same or similar: race, gender, sex, and/or age as the test subject. The determination of aberrant insulin production in a given subject may be achieved using standard tests known in the art. In some embodiments, more than a 2-, 3-, 4-, 5-, 6-, 7-, 8-, 9- or 10-fold increase change in the expression level of a given cell-free (i.e. extracellular, circulating) microRNA signature in a sample from the subject tested as compared to the control is indicative of loss of beta-islet cell number and/or function, and a consequent indication of reduced insulin production capacity in the subject.

The expression of cell-free microRNA signatures may be tested in a biological sample from the subject. Typically, the biological sample will have any cells containing nuclear material (if present) removed prior to determining microRNA expression, preferably without lysing the cells beforehand to ensure that the microRNA measured was predominantly/substantially extracellular in the subject as opposed to intracellular. The biological sample may be a bodily fluid non-limiting examples of which include whole blood, urine, sputum, saliva, synovial fluid, and cerebrospinal fluid. In some embodiments, the biological sample is whole blood, or a separated component of whole blood (e.g. plasma, serum).

The subject from which the biological sample is derived may be a mammalian subject, such as, for example, a human or a non-human mammal. The human subject may be, for example, a Caucasian, an Asian, an African, or a Hispanic. The subject may be of any age.

### Kits

Disclosed herein are kits for performing the methods of the present invention. The kits may be fragmented kits or combined kits. The kits may comprise reagents sufficient for determining the level of expression of a given microRNA signature disclosed herein.

The kits may comprise primers, probes, and/or binding agents for detecting expression of any one or more of the following microRNA/s, in any combination: miR-125b, miR-127, miR-125a-5p, miR-24, miR-29a, miR-27b, miR-99b, miR-181a, miR-223, miR-222, miR-26a, miR-375, miR-30c, miR-374, miR-30b, miR-15b, miR-103, let-7e, miR-7, miR-92a, miR-93, miR-146a, miR-16, miR-200a, miR-26b, miR-186, miR-25, miR-30a-5p, miR-20a, miR-145, miR-199a-3p, miR-148a, miR-30e-3p, miR-22-5p, miR-21, miR-27a, snRNA-U6, miR-152 and miR-34a. For example, the kits may comprise primers, probes, and/or binding agents for detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of these microRNAs.

In some embodiments, the kits may comprise primers, probes, and/or binding agents for detecting miR-21, miR-223, miR-24, miR-26a, miR-29a and miR-326.

In other embodiments, the kits may comprise primers, probes, and/or binding agents for detecting miR-125b, miR-127, miR-125a-5p, miR-24, miR-29a, miR-27b and miR-99b.

In other embodiments, the kits may comprise primers, probes, and/or binding agents for detecting miR-409-5p, miR-340#, miR-301b, miR-155, miR-220c, miR-558, miR-625, miR-9, miR-188, miR-126, miR-210 and/or miR-326.

In still other embodiments, the kits may comprise primers, probes, and/or binding agents for detecting expression of any one or more of the following microRNA/s, in any combination: miR-375, miR-24, miR-26a, miR-27a, miR-27b, miR-29a, miR-146a, miR-155, miR-199a-3p, miR-15b, miR-30b, miR-30c, miR-30e-3p, miR-145, let-7e, miR-222, miR-127, miR-20a, miR-99b, miR-26b, miR-374, miR-223, miR-409-5p, miR-340#, miR-301b, miR-22-5p, miR-186, miR-21, miR-220c, miR-558, miR-625, miR-9, miR-103, miR-125a-5p, miR-125b, miR-148a, miR-16, miR-188, snRNA-U6, miR-7, miR-92a, miR-126, miR-25, miR-152, miR-200a, miR-210, miR-181a, miR-30a-5p, miR-34a, miR-93 and miR-326. For example, the kits may comprise primers, probes, and/or binding agents for detecting 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 of these microRNAs.

In still other embodiments, the kits may comprise primers, probes, and/or binding agents for detecting expression of five or more, six or more, seven or more, eight or more, nine or more, or ten or more, extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93.

In still other embodiments, the kits may comprise primers, probes, and/or binding agents for detecting expression of five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, or twenty-three or more, microRNAs selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

In still other embodiments, the kits may comprise primers, probes, and/or binding agents for detecting expression of five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more, microRNAs selected from the group consisting of: miR-16, miR-20a, miR-21, miR-24, miR-25, miR-26a, miR-26b, miR-30a-5p, miR-30b, miR-30c, miR-92a, miR-93, miR-126, miR-146a, miR-186, miR-199a-3p, miR-222, miR-223, miR-340 and miR-27a.

In still other embodiments, the kits may comprise primers, probes, and/or binding agents for detecting expression of five or more, six or more, seven or more, eight or more, nine or more, ten or more, eleven or more, twelve or more, thirteen or more, fourteen or more, fifteen or more, sixteen or more, seventeen or more, eighteen or more, or nineteen or more, microRNAs selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

In still other embodiments, the kits may comprise primers, probes, and/or binding agents for detecting expression of five or more, six or more, seven or more, eight or more, nine or more, ten or more, eleven or more, twelve or more, thirteen or more, fourteen or more, fifteen or more, sixteen or more, seventeen or more, eighteen or more, or nineteen or more, twenty or more, twenty-one or more, twenty-two or more, twenty-three or more, twenty-four or more, twenty-five or more, twenty-six or more, twenty-seven or more, twenty-eight or more, or twenty-nine or more, microRNAs selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223.

In still other embodiments, the kits may comprise primers, probes, and/or binding agents for detecting expression of five or more, six or more, seven or more, eight or more, nine or more, ten or more, eleven or more, twelve or more, thirteen or more, fourteen or more, fifteen or more, sixteen or more, seventeen or more, eighteen or more, or nineteen or more, twenty or more, twenty-one or more, twenty-two or more, twenty-three or more, twenty-four or more, twenty-five or more, twenty-six or more, twenty-seven or more, twenty-eight or more, or twenty-nine or more, microRNAs selected from the group consisting of: 26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

Additionally or alternatively, the kits may comprise means for extracting RNA from a biological sample.

Additionally or alternatively, the kits may comprise means for reverse-transcribing RNA into cDNA and optionally means for amplifying cDNA. The means for amplifying cDNA may facilitate real-time quantification of the cDNA.

Additionally or alternatively, the kits may comprise control standards to allow normalisation of microRNA signature expression data and/or comparison of microRNA signature expression data to determine whether expression of the microRNA signature is increased, reduced, or in a normal/standard range.

Additionally or alternatively, the kits may comprise buffers, washing reagents, and/or RNAse inhibitors, precipitators (such as glycogen), nuclease-free water and salt solutions required to carry out optimal processing of the sample.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific Example(s), which should not be construed as in any way limiting.

### Example One: Experimental Procedures

The following protocols were employed to generate the experimental results provided in the Examples below:

### (a) Biofluid RNA Isolation

Reagents
1) Nuclease-free 2 ml microcentrifuge tubes (Axygen, MCT-200-C)
2) Glycogen (nuclease-free) 10 μg/μl (Sigma)
3) TRIzol (Ambion, 15596018)
4) Chloroform (Sigma, 2432-500 ml)
5) Isopropyl alcohol, IPA (Sigma, 59304-1L-F)
6) 100% ethanol (Sigma E7023-500 ml)
7) Fresh aliquot for nuclease-free water
8) P10, P20, P200 and P1000 filtered tips
9) RNase AWAY (highly preferred but not essential)
   All plastic ware should be nuclease-free. Never stick your hands into containers of nuclease-free plastic ware. Always pour out whatever is required.

Equipment
1) Refrigerated centrifuge for Eppendorf tubes set at 4° C.
2) Vortex mixer
3) Well calibrated micropipettes designated for (pre-PCR) nucleic acid isolation

Reagent Setup
1) Allow samples to thaw thoroughly on ice prior to commencing.
   a. Due to the time involved in each stage of processing, a maximum of 8 samples should be processed at any time by a single user.
2) Set temperature on a centrifuge to 4° C.
   a. Run “Fast Temp” program if changing rotor or a quick cool down is required.
   b. Use a dedicated centrifuge with the appropriate rotor.
3) Clean gloves with RNase Away prior to commencing.

Procedure
1) Mix sample by gently pipetting up and down, then take out 200 □l* into a 2 ml microcentrifuge tube.
2) Vortex and briefly spin glycogen (20 mg/ml stock) to mix, then add 1 μl to each sample.
3) Add 1 ml of Trizol to each tube. Yellow globules appear in the solution after adding TRIzol. These dissipate following the vortexing and do not seem to affect downstream processing.
4) Vortex each tube for 20 seconds.
5) Incubate at RT (22-24° C.) for 10 minutes.
6) Add 5 μl of 50 nM ath-miR-172a spike in control to each tube, then vortex for 5 secs.
7) Add 200 μl of chloroform to each tube and shake vigorously for 40 seconds. Place the tubes on a rack and ensure each tube is secured properly with your hand. This ensures proper mixing of Trizol and chloroform after addition. Note: DO NOT vortex the tubes at this stage.
8) Incubate at RT for 15 minutes.
9) Centrifuge 12,000×g for 15 minutes at 4° C.
10) Transfer 600-800 μl of the upper aqueous (clear) phase to a fresh 2 ml microcentrifuge tube. Use a 200 μl pipette and tips to remove this gradually. Note: There will be a large amount of white precipitate at the interface between the aqueous and organic layer. Do not disturb this layer. After the removal of 800 μl there will still be some aqueous layer left; try to remove as much as an aqueous layer that you can without any contamination.
11) Add 1.1 ml of IPA to each tube in 2×550 μl aliquots. Note: It is normal that the tubes will be very full at this stage. Be careful to label tops and have clean (dry) fingers to avoid accidental erasure of labels.
12) Vortex each tube for 5 seconds.
13) Incubate at RT for 10 minutes.
14) Centrifuge at 12,000×g for 8 minutes at 4° C. A pellet may not be visible at this stage, so always orient the tubes with the hinge facing outwards to allow estimation of the pellet location—in the bottom towards the hinge.
15) Prepare a fresh volume of 75% ethanol while the samples are spinning. For 8 samples, add 6.75 ml of 100% ethanol to 2.25 ml of nuclease-free water (9 ml in total)
16) Carefully aspirate and discard the supernatant by placing the pipette tip along the wall of the tube opposite to the hinge. When removing the supernatant, reduce the size of the pipette as you go to minimise disturbance to the pellet.
17) Add 1 ml of freshly-prepared 75% ethanol to each tube and invert 5 times. If a pellet is visible, you will see it floating after inversion.
18) Centrifuge at 7,500×g for 5 minutes at 4° C.
19) Carefully remove and discard supernatant. As before (step 16), reduce the volume of the pipette when removing the supernatant.
20) Allow the tubes to dry at RT for 5 minutes. Do not over dry the samples as this will reduce the solubility of the RNA. If you have left excess ethanol and are not confident in removing it without disturbing the pellet, you may need to incubate tubes at 37° C. for maximum of 3 minutes. Any longer can damage RNA quality.
21) Add 10-15 μl of nuclease-free water to each tube and resuspend. Always store RNA on the ice after this stage. The volume may vary depending on the pellet size.
22) Measure concentration of RNA using Nanodrop. The 260/280 ratio may be as low as 1.3 but this does not affect downstream processing. If you are not going to proceed with downstream analysis immediately, then skip this step and proceed to store your RNA. Always measure (Nanodrop, Qubit or Bioanalyzer) before downstream processing.
23) Store RNA at −80° C. Always log your sample details in the −80° C. freezer log on the networked lab drive.

### (b) Automated RNA Isolation

Reagents
1) Collection microtubes (Qiagen, 19560)
2) Lids for collection microtubes (Qiagen, 19566)
3) Glycogen (nuclease-free) 10 μg/μl (Sigma)
4) TRIzol (Ambion, 15596018)
5) Chloroform (Sigma, 2432-500 ml)
6) Isopropyl alcohol, IPA (Sigma, 59304-1L-F)
7) 50 nM ath-miR-172a (8-well strips, stored in −80° C. freezer)
8) RNeasy 96 QIAcube HT kit (Qiagen, CAT 74171)
9) QIAcube HT Plasticware (Qiagen, CAT 950067)
10) Tip disposal box (Qiagen, CAT 990550)
11) Reagent trough with lid, 70 ml (Qiagen, CAT 990554)
12) Reagent trough with lid, 170 ml (Qiagen, CAT 990556)
13) Filter tips, OnCor C, 200 μl (Qiagen, CAT 990610)
14) P10, P20, P200 and P1000 filtered tips
15) RNase AWAY (highly preferred but not essential)
   NB: All plasticware should be nuclease-free. Never stick your hands into containers of nuclease-free plastic ware. Always pour out whatever is required.

Equipment
1) QIAcube HT
2) Refrigerated centrifuge for plates set at 4° C.
3) Vortex mixer
4) Well calibrated micropipettes designated for (pre-PCR) nucleic acid isolation

Reagent Setup
1) Allow samples to thaw thoroughly on ice prior to commencing.
2) Clean gloves with RNase Away prior to commencing.
   Procedure
1) Mix sample by gently pipetting up and down, then aliquot 10011 into one collection microtube (96 racked).
2) Vortex and briefly spin glycogen (10 μg/μl stock) to mix, then add 11 to each sample.
3) Add 500 μl of Trizol to each tube. Yellow globules appear in the solution after adding Trizol. These dissipate following the vortexing and do not seem to affect downstream processing. Wear appropriate PPE and be careful when vortexing or shaking tubes containing Trizol.
4) Vortex the microtubes for 40 secs or until the yellow globules disappear.
5) Incubate at RT (22-24° C.) for 10 minutes.
6) Briefly centrifuge (1 min, 1500 rpm) the racked microtubes at 4° C. to remove Trizol from the caps.
7) Add 2.5 μl of 50 nM ath-miR-172a spike in control to each tube. One 8-well strip contains enough for an entire 96-well plate.
8) Add 100 μl of chloroform to each tube and shake vigorously for 40 seconds. Secure the caps using an upside down plastic lid and elastic bands as shown below.
   NB: DO NOT vortex the tubes at this stage.
9) Incubate at RT for 15 minutes.
10) Centrifuge 3,200×g for 25 minutes at 4° C.
11) Turn on QIAcube HT system. Remove tip eject cover (red). Open the pre-treatment run file.
12) Place microtubes into position C1 of the QIAcube HT. The trough holder in this position must be removed first.
13) Load an empty S-block into position BI. Load tips.
14) Begin the run. This program will transfer 300 μl of aqueous phase into the S-block.
15) Remove the microtubes. For protein/DNA analysis, store these samples at −80° C. Place the trough holder back into this position.
16) Open the purification run file.
17) Load the appropriate reagents in their respective troughs. See software for specific volumes. Ensure there is a little excess volume to avoid running out mid-cycle.
18) Load RNeasy plate and Elution plate in A1 (left/right respectively). Ensure that A1 is top left. The left section of A1 is for waste, while the right is for elution.
19) Load tips (2×96). Start the run and complete the pre-run checklist.
20) Remove the Elution plate. Cap these tubes and then store at −80° C. Always log your sample details on the networked database.
21) Remove the reagent troughs and discard remaining liquid. All troughs, except the Top Elute, may be rinsed with nuclease-free water and left to dry. The Top Elute trough must be wiped out with a Kim wipe. Reagent troughs can be used for a maximum of one month or one full RNeasy kit before being discarded.
22) Remove channel block (three pieces) and pour 20 ml of RO water down the waste chute. Replace the tip eject cover (red). Ensure that the rubber gasket is removed from the filter carriage for cleaning.
23) Soak channel block in 1% Trigene for 15-30 mins. Rinse with RO water. Dry.
24) Run cleaning cycle, including UV if this is the last run of the day. Select to turn off after the cleaning cycle is completed.

### (c) Open Array Low Sample Input

Reagents
1) Custom OpenArray Slides (Applied Biosystems, 4470813)
2) Custom RT primers (included with custom slides)
3) Custom PreAmp primers (Applied Biosystems, 4441856)
4) TaqMan microRNA reverse transcription kit (Applied Biosystems, 200 r×n 4366596, 1000 r×n 4366597)
5) 15 nM ath-miR-159a synthetic miRNA (Sigma)
6) OpenArray 384-well sample plate (Applied Biosystems, 4406947)
7) Aluminium Seal (Beckman-Coulter, 538619)
8) TaqMan PreAmp mastermix (Applied Biosystems, 1 ml 4391128, 5 ml 4488593)
9) TE Buffer (Invitrogen, 12090015)
10) OpenArray accessories kit (Applied Biosystems, 4453975)
11) TaqMan OpenArray real-time PCR mastermix (Applied Biosystems, 1.5 ml 4462159, 5 ml 4462164)
12) AccuFill tips (Applied Biosystems, 1 box 4457246, 10 boxes 4458107)
13) Filtered pipette tips
14) Nuclease-free water (Qiagen, 129117)
15) 0.2 ml 96-well plates (Axygen, PCR-96M2-HS-C)
   NB: All plastic ware should be nuclease-free. Never stick your hands into containers of nuclease-free plastic ware. Always pour out whatever is required.

Equipment
1) Thermocycler
2) QuantStudio™ 12K Flex Accufill System
3) QuantStudio™ 12K Flex Real-Time PCR System
4) Vortex
5) Centrifuge
6) Axymat Silicon Seals (Axygen, AM-96-PCR-RD)

Reagent Setup
1) Download the relevant run file/s (.tpf) from the Life Technologies website (https://www.thermofisher.com/au/en/home/products-and-services/product-types/download-openarray-tpf-and-spf-plate-files.html) using the lot and a serial number of the slide.
2) Thaw all reagents on the ice, except for the RT enzyme, which must remain at −20° C. until use.

Procedure

Pt 1: Reverse Transcription
1. Dilute the RNA samples to <10 ng/μl. This protocol is designed for samples with low RNA concentrations. Diluting to <10 ng/μl (around 8.5 ng/μl is sufficient) allows for >1 μl to be taken in the next step, increasing accuracy.
2. Add 10 ng of RNA to the respective well of a 96-well plate and then bring the volume to 3 μl.
3. Create an RT master mix using the components from the reverse transcription kit, as detailed below. It is recommended to add 5% excess to account for pipette error.

| | Per reaction |
|---|---|
| Component | (μl) |
| Custom RT Primers | 0.75 |
| dNTPs with dTTp (100 mM) | 0.15 |
| RT Buffer (10X) | 0.75 |
| MgCl₂ (25 mM) | 0.9 |
| RNase Inhibitor (20 U/μl) | 0.09 |
| Nuclease-free Water | 0.25 |
| ath-miR-159a Spike In (15 nM) | 0.10 |
| Multiscribe Reverse Transcriptase (50 U/μl) | 1.5 |
| Total | 4.5 |

4. Pipette to mix and then centrifuge briefly at 10,000×g for 10 sec (quick spin).
5. Aliquot 4.5 μl of the mastermix into each well. Seal with a silicon seal.
6. Invert to mix and then quick spin.
7. Incubate on ice for 5 min.
8. Place into the thermocycler and run the following program: 40 cycles (16° C. for 2 min, 42° C. for 1 min, 50° C. for 1 sec), 85° C. for 5 min, hold at 4° C.
9. cDNA can be stored at −20° C. or used immediately.

Pt 2: Preamplification
10. Create a preamp mastermix using the components listed below. Swirl the PreAmp mastermix prior to use. It is recommended to add 5% excess to account for pipette error.

| | Per reaction |
|---|---|
| Component | (μl) |
| TaqMan PreAmp Mastermix (2X) | 20 |
| Custom PreAmp Primers (10X) | 4 |
| Nuclease-free water | 8.5 |
| Total | 32.5 |

11. Invert to mix, and then quick spin.
12. Aliquot 32.5 μl of the mastermix into each well.
13. Invert to mix, and then quick spin.
14. Incubate on ice for 5 mins.
15. Place into thermocycler and run the following program: 95° C. for 10 mins, 55° C. for 2 mins, 72° C. for 2 mins, 16 cycles (95° C. for 15 secs, 60° C. for 4 mins), 99° C. for 10 mins, hold at 4° C.
16. Invert preamplified cDNA and then quick spin.
17. Dilute 1:20 by adding 4 μl of preamplified cDNA to 76 μl of 0.1×TE buffer.
18. Both diluted and undiluted preamplified cDNA can be stored at −20° C. for up to 1 week or used immediately.

Pt3: Loading OpenArray Slides and Performing qPCR
19. Combine 5 μl of diluted, preamplified cDNA to 5 μl of TaqMan OpenArray real-time PCR mastermix in a new 96-well plate. Seal with a silicon seal.
20. Vortex and quick spin.
21. Aliquot 5 μl of each sample into 1 well of the 384-well sample plate. The position of each sample will depend on the configuration of the custom slide. Each well of the sample plate corresponds to one subarray of the OpenArray slide. An entire slide will take 48 wells (4 rows, 12 columns).
22. Seal the samples plate. It is advisable to pre-cut the seal into the required sections, so the sections may be sealed/unsealed individually to reduce evaporation. Alternatively, the plate may be sealed with an intact seal, and then sections can be individually cut out when loading.
23. Centrifuge the sample plate at 490×g for 1 min at 4° C. Load the OpenArray slides within 1 hr.
24. Remove the required slides from the freezer and allow them to come to room temperature (˜15 mins). As these slides work on hydrophobic/hydrophilic interactions, condensation should be avoided.
25. Set up consumables from the accessory kit.
   25.1. Gently pull on the plunger of the immersion fluid syringe to loosen. Remove cap, place tip on and flush air from the tip.
   25.2. Removed slide lid and plug from packaging.
   25.3. Place the loading system tips within the machine and remove the lid.
   25.4. Place sample plate within PCR system.
   25.5. Put gloves on. Use a size lower than normal to minimise the risk of accidentally marking the slide lid.
   25.6. Carefully open slide packaging. Slowly tip slide into the hand. Do not touch the top of the slide. Place slide into the AccuFill, with the barcode on the left.
   25.7. Remove seal from the portion of the sample plate intended for loading. Use the loading system software to enter the slide barcode, slide position, sample position and tip configuration.
26. When all relevant checks are completed, press load slide. While the PCR system is loading the slide, remove the clear and red plastic from the bottom of the slide lid. When finished loading, carefully remove and seal the slide within 90 sec.
   26.1. Place the slide within the plate clamp. Place the slide lid onto the slide. Clamp for 30 sec. Ensure the lid is positioned so that barcode is correctly displayed. Remove the assembly from the plate clamp.
   26.2. Position immersion fluid syringe within the slide so that the tip is pressing against the lid. Slowly fill a slide with immersion fluid, ensuring the fluid runs along the lid. Once full, seal the slide with the plug, turning the screw until the handle breaks off.
   26.3. Remove the plastic cover on the top of the slide lid, and then carefully place into the slide carrier of the real-time PCR system. Ensure there is support on the bottom of the slide as it is being lowered, so it does not drop suddenly, and do not touch the top of the slide. It is OK to touch the sides of the slide/cassette.
27. Initialise the QuantStudio 12K Flex and start the qPCR run.
   27.1. Select “OpenArray” within the PCR-system software. Press “Find Slide IDs”. This will take a few mins. If the software cannot find the plate ID, it will ask for it to be entered manually.
   27.2. Press “Confirm Plate Centres”. Again, this will take a few mins. Check that the red dot is within the centre and that there are no fingerprints/marks on the top of the slide. Load the respective tpf file for each slide and specify a result file name and location. Press “Start Run”. The program will take approximately 2 hr to complete.

Data obtained from these custom microRNA panels was assessed to remove non-specific amplification (results with an AMP score of <1.24 and/or a Cq confidence score of <0.6 were omitted). Data were normalised to the RNA isolation and RT ath-miR spike-in controls and transcript abundance was calculated using the fold over detectable method (limit of detection=39) described earlier (Hardikar A et al. 2014 J Am Heart Assoc).

(d) Human Islet Cell Death Assay

Reagents
1) Sterile 1.7 ml microcentrifuge tubes (Axygen, MCT-175-C)
2) Sterile 15 ml tubes (Falcon, FAL352096)
3) Ethanol (70%) for disinfection
4) Sodium Nitroprusside (SNP) (Sigma, 71778)
5) CMRL media (no glutamine) (Gibco, 11530037)
6) CMRL media+2% bovine serum albumin, BSA (Roche, 10775835001)+2×GlutaMAX* (Gibco, 35050-061)
7) Dulbecco's phosphate buffered saline, DPBS (Gibco, 14190-250)
8) P10, P20, P200 and P1000 autoclaved tips
9) P1000 autoclaved tips with the end cut off
10) 10 ml serology pipette tips
11) 24 well cell suspension plates
12) 0.2 μm syringe filter
13) 2 ml syringe

Equipment
1) Water bath set at 37° C.
2) Centrifuge with 15 ml swing-bucket rotor
3) Microcentrifuge
4) Well calibrated micropipettes designated for cell culture
5) Electronic serological pipette

Reagent Setup
1) Set water bath to 37° C.
   a. Place PBS and CMRL+2% BSA+2×GlutaMAX into the water bath for >15 mins
2) Label 1.7 ml Eppendorf tubes
   a. One per well for the islet cell pellet
   b. Three per well for the supernatant aliquots
3) Clean gloves, and anything else entering the biosafety cabinet, with 70% ethanol prior to use

Procedure

Pt 1: Plate Setup
1) Prepare a fresh stock solution of 40 mM SNP by dissolving the SNP crystals in PBS. 0.0596 g SNP in 5 ml PBS. Vortex to dissolve crystals.
2) Dilute SNP to 20 mM in CMRL (nothing added).
3) Sterilise 20 mM SNP solution by passing it through a 0.2 um syringe filter. Remove the plunger from the syringe, attach a filter, pour in 20 mM SNP solution, and then push the liquid through using the plunger. Ensure there is a sterile tube to collect the sterilised solution. It is also recommended to pre-wet the syringe filter by passing through a small amount of CMRL media to avoid binding SNP to the filter.
4) Serially dilute the SNP solution in sterile CMRL (nothing added). This should be completed as 1:10 dilutions to make 2 mM, 200 μM, 20 μM and 2 μM SNP.
5) Aliquot 500 ul of each SNP dilution into the respective well of a 24-well suspension plate. It is recommended to perform three technical replicates. If you are intending to perform staining or FACS on these cells, create additional replicates.
6) Place the plate in an incubator at 37° C. with 5% CO₂. This will warm the media prior to the addition of the islets.

Pt 2: Islet Addition
7) Place islets into a 15 ml Falcon tube and centrifuge at 1500 rpm for 2 min. Discard supernatant.
8) Resuspend islets in 10 ml warm PBS.
9) Centrifuge at 1500 rpm for 2 mins. Discard supernatant.
10) Repeat steps 8 and 9.
11) Resuspend islets in CMRL+2% BSA+2×GlutaMAX. Add an appropriate volume of media so 200 IEQs will be added in 500 ul of media, with 5-10% extra to account for pipette error.
12) Gently mix islets and then add 500 ul of the islet suspension to each well of the SNP plate. Use the P1000 pipette tips with the end cut off to avoid shearing the islets. It is very important to mix the islets between every aliquot. It is also recommended to add the islets across the SNP dilutions rather than adding to all replicates of one condition before moving on to the next. This will evenly spread out any variation among all of the conditions.
13) Place the plate into the incubator at 37° C. with 5% CO₂ for 24 hours. Take note of the time when the plate was placed in the incubator.

Pt 3: Islet and Supernatant Harvest
14) Gently mix the islets and media, and then transfer to a sterile 1.7 ml Eppendorf tube. Use the P1000 pipette tips with the end cut off to avoid shearing the islets.
15) Centrifuge at 2000 rpm for 2 mins. Always orient the tubes with the hinge facing outwards to allow estimation of the pellet location—in the bottom towards the hinge.
16) Carefully aspirate 900 ul of the supernatant into a fresh 1.7 ml Eppendorf tube by placing the pipette tip along the wall of the tube opposite to the hinge. Place the supernatant on the ice at this stage.
17) Mix supernatant by pipetting 5 times, and then Aliquot 300 ul in two additional 1.7 ml Eppendorf tubes.
18) Centrifuge the tubes containing the islets at 2000 rpm for 2 mins. Always orient the tubes with the hinge facing outwards to allow estimation of the pellet location—in the bottom towards the hinge
19) Carefully aspirate and discard the remaining supernatant by placing the pipette tip along the wall of the tube opposite to the hinge.
20) Store the supernatant aliquots and islet pellets at −80° C. Always log your sample details in the −80° C. freezer log on the networked lab drive.

(e) Mouse Terminal Blood Collection and Dissection

Reagents
1) 1.7 ml microcentrifuge tubes (Axygen, MCT-175-C)
2) 0.1 M EDTA, pH 7.5-8
3) Dulbecco's Phosphate Buffered Saline, DPBS (Gibco, 14190-250)
4) 4% paraformaldehyde, PFA
5) 21G needles
6) 2 ml syringes
7) Liquid nitrogen or dry ice
8) Wet ice
9) 70% Ethanol for disinfection
10) Isofluorane
11) Blood glucose testing strips

Equipment
1) Isofluorane anaesthetising equipment
2) Secured nose cone
3) Calibrated blood glucose testing meter
4) Sterilised dissection equipment (160° C. overnight/200° C. for >2 hours)
5) Working area with absorbent mat

Setup
1) Mice should be fasted for 12-14 hours overnight prior to this procedure.
2) Weigh and record the tubes containing DPBS.
3) Attach a needle to a syringe for each mouse to be processed.
4) Ensure there is a different set of scissors and forceps for each tissue, kept in separate, labelled positions.
5) Aliquot the following reagent into separate 1.7 ml tubes as necessary:
   a. 10 μl 0.1M EDTA pH7.5-8 (1 per mouse)
   b. 500 μl DPBS (1 per mouse)
   c. 500 μl 4% PFA (1 per tissue)
   Procedure
1. Weigh mouse. Record their weight on the Record Sheet.
to 2. Anaesthetise the mouse using 3% isofluorane with 0.5 L/min oxygen. Ensure that reflexes are absent before moving to the next step.
3. Transfer mouse to the working area (laying it on its back) and continue to anaesthetise using 1.5-2% isofluorane administered through the nose cone.
4. Disinfect the abdomen with 70% ethanol.
5. Collect blood via cardiac puncture. Insert a 21 G needle under the ziphoid process (base of the sternum), parallel to the mouse's body, angled slightly to the right (mouse's left). Once blood is seen in the syringe, gently pull back on the plunger.
6. Euthanise the mouse via cervical dislocation. Turn off the isofluorane.
7. Remove the needle from the syringe and then slowly dispense the blood into the tube containing 0.1M EDTA (pH7.5-8). Place used needle in the appropriate sharps container.
8. Invert to mix and then place on ice.
9. Measure the blood glucose using the small amount of blood left in the syringe.
10. Disinfect the abdomen again using 70% ethanol.
11. Using scissors, cut away the skin to expose the peritoneum. Use forceps to pinch the skin away from the peritoneum, create a small incision, and then separate the skin from the peritoneal membrane. This minimises fur contamination.
12. Open the peritoneum, being careful not to cut any of the underlying organs.
13. Identify the pancreas and carefully remove the organ. The pancreas is located on the right side (left side of the mouse) and is easily located by gently pulling on the dark red spleen. The pancreas is attached to several points of the spleen, stomach, and duodenum; these points must be carefully cut away to release the pancreas.
14. Place the pancreas into the tube containing DPBS, weigh the tube, and then record this measurement. This will allow measurement of the pancreas weight.
15. Remove the pancreas from the DPBS, gently remove excess liquid by dabbing it onto some tissue paper and then store appropriately. If the pancreas is needed for RNA/DNA isolation, place into an empty tube and snap freeze using dry ice or liquid nitrogen. If it is required for staining, store in 4% paraformaldehyde and place on wet ice. If both are required, use a sterile scalpel to cut the pancreas lengthwise prior to storage.
16. Remove any other tissues that are required and store appropriately.
17. Discard the carcass by placing it into the provided plastic bag and store it in the animal house freezer for disposal.
18. Centrifuge the blood at 2000×g for 20 mins at 4° C.
19. Aliquot the plasma into fresh 1.7 ml tubes.
20. You may isolate RNA directly from the fresh plasma, or store the aliquots at −80° C. Always log your sample details in the −80° C. freezer log on the networked lab drive.
21. Store snap-frozen tissues at −80° C. Always log your sample details in the −80° C. freezer log on the networked lab drive.
22. Store tissues in 4% PFA at 4° C.

(f) Immunofluorescent Staining of Paraffin-Embedded Tissues SOP

Reagents:
1) Normal donkey serum (Thermo Fisher, 14190250)
2) 100% ethanol (Sigma, E7023-500 ml)
3) Distilled (or MilliQ) water
4) Tissue paper or Kimwipes
5) Xylene (Thermo Fisher, AJA2342-5L)
6) Primary antibody/ies
7) Dulbecco's phosphate buffered saline, DPBS (Gibco, 14190-250)
8) Secondary antibody/ies
9) Vectashield (Vector Laboratories, H-1000)
10) Hoechst 33342 nuclear stain (10 mg/ml)
11) Coverslips
12) Nail polish
   NB: All plastic ware should be nuclease free. Never stick your hands into containers of nuclease-free plastic ware. Always pour out whatever is required.

Equipment
1) Convection oven set at 85-90° C.
2) Coplin Jars
3) Forceps
4) Hydrophobic marker
5) Moist chamber

Reagent Setup
1) Fill two Coplin jars with Xylene.
2) Fill four Coplin jars decreasing concentrations of ethanol in this order: 100%, 90%, 70% and 50% (50 ml in distilled water).
3) Fill one Coplin Jar with distilled water.
4) Dilute normal donkey serum (NDS) with dPBS to 4% NDS
5) Dilute primary antibodies to a working stock in 4% NDS. The actual dilution factor with depend on the antibody used [for example the Guinea Pig anti-insulin polyclonal (from DAKO, catalogue number-A0564(01)) insulin is usually 1:100]. Multiple antibodies can be combined into one stock ONLY if they were raised in different animals.
6) Dilute secondary antibodies 1:100 to a working stock in 4% NDS. Ensure the antibodies target the animal in which the primary antibody was raised
7) Set up moist chamber by placing moist paper towel within the chamber.
8) Create the mounting solution by adding 10 μl of Hoechst to 1 ml of Vectashield.

Procedure

Pt 1: Primary Antibody
1. Place slide at 85-90° C. for 2-5 mins or until the paraffin wax becomes translucent (check every minute after the first 2 minutes). The tissue will still be opaque.
2. Immediately place slide into the first xylene Coplin jar for 2-3 mins. When adding the slide to a Coplin jar, gently wash the slide by dipping into the liquid 3-4 times.
3. Transfer slide to the second xylene Coplin jar for 5 mins. When transferring the slide between jars, gently dab the edge of the slide onto tissue paper or Kimwipes to remove excess liquid.
4. Transfer the slide to 100% ethanol for 5 mins.
5. Transfer the slide to 90% ethanol for 5 mins.
6. Transfer the slide to 70% ethanol for 5 mins.
7. Transfer the slide to 50% ethanol for 5 mins.
8. Transfer the slide to distilled water for 5 mins.
9. Remove slide and dab off excess liquid.
10. Using the hydrophobic marker, draw around your tissue section. Ensure that the line is close to your sample without touching it.
11. Add enough 4% NDS to cover the section.
12. Place slide into the moist chamber and incubate at room temperature for 20 mins.
13. Tilt the slide and use a pipette to remove and discard the liquid. Do not remove the slides from the moist chamber.
14. Add enough of the selected primary antibody (working stock in 4% NDS) to cover the tissue.
15. Seal the moist chamber with parafilm and incubate at 4° C. overnight.

Pt 2: Secondary Antibody
16. Tilt the slide and use a pipette to remove and discard the primary antibody.
17. Add DPBS and incubate at room temperature for 3-5 mins. Add as much PBS as possible within the confines of the hydrophobic marker. This will create a large bubble of fluid over the tissue section.
18. Remove liquid.
19. Repeat steps 17 and 18 at least 4 more times.
20. Add enough secondary antibody (working stock diluted in 4% NDS) to cover the step, and all subsequent steps MUST be completed in the dark to ensure that the reporter dyes to not degrade.
21. Seal the moist chamber with parafilm and incubate at 37° C. for 1 hour. Ensure there is ample liquid in the tissue paper used in the moist chamber.
22. Remove the secondary antibody and then repeat steps 17 and 18 at least five times to remove any unbound secondary antibodies.
23. Tilting the slide and place folded tissue or Kimwipe at the bottom of the sample to soak up the remaining PBS. It is important not to touch the tissue section or wipe the liquid.
24. Add 20 μl of the mounting solution (Vectashield+Hoechst).
25. Add a coverslip and seal using nail polish. Place one edge of the coverslip to the side of the sample, in contact with the mounting solution. Slowly lower the other edge using a scalpel blade (or another suitable thin, flat utensil), allowing the solution to spread along the coverslip. This method minimises the introduction of bubbles.
26. Store slide(s) at RT in the dark until the slide is dry (˜30 minutes).
27. Examine slide(s) using a fluorescent microscope.
28. Place the slide(s) at 4° C. for long-term storage.

(g) Flow Cytometry Cell Death Analysis

Reagents:
1) Sterile 15 ml Falcon tubes
2) Ethanol (70%) for disinfection
3) Accutase (Merk Millipore)
4) CMRL media+10% FCS (Gibco)
5) Dulbecco's phosphate buffered saline (DPBS) (Gibco)
6) P10, P20, P200 and P1000 autoclaved tips
7) P1000 autoclaved tips with the end cut off (to transfer islets only)
8) 10 ml serology pipette tips
9) FACS tubes
10) Cell strainer (70 μm)
11) Hypotonic buffer—see step 1.

Equipment
1) Water bath set at 37° C.
2) Centrifuge with 15 ml swing-bucket rotor
3) Well calibrated micropipettes designated for cell culture
4) Electronic serological pipette
5) Flow-cytometer (turned on >20 mins before use)

Reagent Setup
1) Set water bath to 37° C.
   a. Place PBS and CMRL+10% FCS into the water bath for >15 mins
2) Clean gloves, and anything else entering the biosafety cabinet, with 70% ethanol prior to use

Procedure
1 Create the hypotonic buffer.

TABLE 1

| | | | Amount | |
|---|---|---|---|---|
| Reagent | Original Conc. | Final Conc. | (for 2 ml) | |
| Sodium Citrate | 1% | 0.1% | 200 | ul |
| Triton-X 100 | 10% | 0.1% | 20 | ul |
| Propidium Iodide | 1 mg/ml | 500 ug/ml | 100 | ul |
| Nuclease-free Water | — | — | 1680 | ul |

2. Transfer cells and media into a 15 ml tube. It is recommended to gently mix the cells prior to transfer.
3. Centrifuge at 1500 rpm for 2 mins.
4. Remove media.
5. Resuspend islets in 5 ml warm DPBS.
6. Repeat washing steps 3-4.
7. Remove DPBS.
8. Add 200 μl of Accutase and incubate in a 37° C. water bath for 10 mins.
9. Gently pipette to break the cells apart.
10. Add 5 ml of CMRL+10% FCS
11. Centrifuge at 1500 rpm for 2 mins.
12. Remove supernatant.
13. Add 5 ml of CMRL+10% FCS.
14. Incubate for 30-60 mins at 37° C. with 5% C02. This will allow the cells to recover
15. Centrifuge at 1500 rpm for 2 mins.
16. Remove media
17. Add 250 μl of hypotonic buffer.
18. Add to FACS tubes. If any clumps can still be seen, pass the liquid through a cell filter as it is added to the FACS tubes.
19. Set up and run FACSCalibur. Read at least 10,000 events.

(b) MicroRNA Utilised in these Studies

TABLE 2

| SEQ ID | Name of the | |
|---|---|---|
| NO | hsa-miRNA/snRNA | Primary microRNA/snRNA sequence |
| 1 | miR-375 | CCCCGCGACGAGCCCCUCGCACAAACCGGACCUGAGCGUUUUGUUCGUUCG |
| | | GCUCGCGUGAGGC |
| 2 | miR-24 | CUCCGGUGCCUACUGAGCUGAUAUCAGUUCUCAUUUUACACACUGGCUCAG |
| | | UUCAGCAGGAACAGGAG |
| 3 | miR-26a | GUGGCCUCGUUCAAGUAAUCCAGGAUAGGCUGUGCAGGUCCCAAUGGGCCU |
| | | AUUCUUGGUUACUUGCACGGGGACGC |
| 4 | miR-27a | CUGAGGAGCAGGGCUUAGCUGCUUGUGAGCAGGGUCCACACCAAGUCGUGU |
| | | UCACAGUGGCUAAGUUCCGCCCCCCAG |
| 5 | miR-27b | ACCUCUCUAACAAGGUGCAGAGCUUAGCUGAUUGGUGAACAGUGAUUGGUU |
| | | UCCGCUUUGUUCACAGUGGCUAAGUUCUGCACCUGAAGAGAAGGUG |
| 6 | miR-29a | AUGACUGAUUUCUUUUGGUGUUCAGAGUCAAUAUAAUUUUCUAGCACCAUC |
| | | UGAAAUCGGUUAU |
| 7 | miR-146a | CCGAUGUGUAUCCUCAGCUUUGAGAACUGAAUUCCAUGGGUUGUGUCAGUG |
| | | UCAGACCUCUGAAAUUCAGUUCUUCAGCUGGGAUAUCUCUGUCAUCGU |
| 8 | miR-155 | CUGUUAAUGCUAAUCGUGAUAGGGGUUUUUGCCUCCAACUGACUCCUACAU |
| | | AUUAGCAUUAACAG |
| 9 | miR-199a-3p | GCCAACCCAGUGUUCAGACUACCUGUUCAGGAGGCUCUCAAUGUGUACAGU |
| | | AGUCUGCACAUUGGUUAGGC |
| 10 | miR-15b | UUGAGGCCUUAAAGUACUGUAGCAGCACAUCAUGGUUUACAUGCUACAGUC |
| | | AAGAUGCGAAUCAUUAUUUGCUGCUCUAGAAAUUUAAGGAAAUUCAU |
| 11 | miR-30b | ACCAAGUUUCAGUUCAUGUAAACAUCCUACACUCAGCUGUAAUACAUGGAU |
| | | UGGCUGGGAGGUGGAUGUUUACUUCAGCUGACUUGGA |
| 12 | miR-30c | AGAUACUGUAAACAUCCUACACUCUCAGCUGUGGAAAGUAAGAAAGCUGGG |
| | | AGAAGGCUGUUUACUCUUUCU |
| 13 | miR-30e-3p | GGGCAGUCUUUGCUACUGUAAACAUCCUUGACUGGAAGCUGUAAGGUGUUC |
| | | AGAGGAGCUUUCAGUCGGAUGUUUACAGCGGCAGGCUGCCA |
| 14 | miR-145 | CACCUUGUCCUCACGGUCCAGUUUUCCCAGGAAUCCCUUAGAUGCUAAGAU |
| | | GGGGAUUCCUGGAAAUACUGUUCUUGAGGUCAUGGUU |
| 15 | let-7e | CCCGGGCUGAGGUAGGAGGUUGUAUAGUUGAGGAGGACACCCAAGGAGAUC |
| | | ACUAUACGGCCUCCUAGCUUUCCCCAGG |
| 16 | miR-222 | GCUGCUGGAAGGUGUAGGUACCCUCAAUGGCUCAGUAGCCAGUGUAGAUCC |
| | | UGUCUUUCGUAAUCAGCAGCUACAUCUGGCUACUGGGUCUCUGAUGGCAUC |
| | | UUCUAGCU |
| 17 | miR-127 | UGUGAUCACUGUCUCCAGCCUGCUGAAGCUCAGAGGGCUCUGAUUCAGAAA |
| | | GAUCAUCGGAUCCGUCUGAGCUUGGCUGGUCGGAAGUCUCAUCAUC |
| 18 | miR-20a | GUAGCACUAAAGUGCUUAUAGUGCAGGUAGUGUUUAGUUAUCUACUGCAUU |
| | | AUGAGCACUUAAAGUACUGC |
| 19 | miR-99b | GGCACCCACCCGUAGAACCGACCUUGCGGGGCCUUCGCCGCACACAAGCUC |
| | | GUGUCUGUGGGUCCGUGUC |
| 20 | miR-26b | CCGGGACCCAGUUCAAGUAAUUCAGGAUAGGUUGUGUGCUGUCCAGCCUGU |
| | | UCUCCAUUACUUGGCUCGGGGACCGG |
| 21 | miR-374 | UACAUCGGCCAUUAUAAUACAACCUGAUAAGUGUUAUAGCACUUAUCAGAU |
| | | UGUAUUGUAAUUGUCUGUGUA |
| 22 | miR-223 | CCUGGCCUCCUGCAGUGCCACGCUCCGUGUAUUUGACAAGCUGAGUUGGAC |
| | | ACUCCAUGUGGUAGAGUGUCAGUUUGUCAAAUACCCCAAGUGCGGCACAUG |
| | | CUUACCAG |
| 23 | miR-409-5p | UGGUACUCGGGGAGAGGUUACCCGAGCAACUUUGCAUCUGGACGACGAAUG |
| | | UUGCUCGGUGAACCCCUUUUCGGUAUCA |
| 24 | miR-340# | UUGUACCUGGUGUGAUUAUAAAGCAAUGAGACUGAUUGUCAUAUGUCGUUU |
| | | GUGGGAUCCGUCUCAGUUACUUUAUAGCCAUACCUGGUAUCUUA |
| 25 | miR-301b | GCCGCAGGUGCUCUGACGAGGUUGCACUACUGUGCUCUGAGAAGCAGUGCA |
| | | AUGAUAUUGUCAAAGCAUCUGGGACCA |
| 24 | miR-340# | UUGUACCUGGUGUGAUUAUAAAGCAAUGAGACUGAUUGUCAUAUGUCGUUU |
| | | GUGGGAUCCGUCUCAGUUACUUUAUAGCCAUACCUGGUAUCUUA |
| 25 | miR-301b | GCCGCAGGUGCUCUGACGAGGUUGCACUACUGUGCUCUGAGAAGCAGUGCA |
| | | AUGAUAUUGUCAAAGCAUCUGGGACCA |
| 26 | miR-22-5p/ | GGCUGAGCCGCAGUAGUUCUUCAGUGGCAAGCUUUAUGUCCUGACCCAGCU |
| | miR-22* | AAAGCUGCCAGUUGAAGAACUGUUGCCCUCUGCC |
| 27 | miR-186 | UGCUUGUAACUUUCCAAAGAAUUCUCCUUUUGGGCUUUCUGGUUUUAUUUU |
| | | AAGCCCAAAGGUGAAUUUUUUGGGAAGUUUGAGCU |
| 28 | miR-21 | UGUCGGGUAGCUUAUCAGACUGAUGUUGACUGUUGAAUCUCAUGGCAACAC |
| | | CAGUCGAUGGGCUGUCUGACA |
| 29 | miR-220c | CACUGGGACCACACAGGGCUGUUGUGAAGACUCAGUGAGCUCAUCCCCACA |
| | | CAGCCUUCAGCACAGGGCCUGGCUCAGGGCAG |
| 30 | miR-558 | GUGUGUGUGUGUGUGUGUGGUUAUUUUGGUAUAGUAGCUCUAGACUCUAUU |
| | | AUAGUUUCCUGAGCUGCUGUACCAAAAUACCACAAACGGGCUG |
| 31 | miR-625 | AGGGUAGAGGGAUGAGGGGGAAAGUUCUAUAGUCCUGUAAUUAGAUCUCAG |
| | | GACUAUAGAACUUUCCCCCUCAUCCCUCUGCCCU |
| 32 | miR-9 | CGGGGUUGGUUGUUAUCUUUGGUUAUCUAGCUGUAUGAGUGGUGUGGAGUC |
| | | UUCAUAAAGCUAGAUAACCGAAAGUAAAAAUAACCCCA |
| 33 | miR-103 | UUGUGCUUUCAGCUUCUUUACAGUGCUGCCUUGUAGCAUUCAGGUCAAGCA |
| | | GCAUUGUACAGGGCUAUGAAAGAACCA |
| 34 | miR-125a-5p | UGCCAGUCUCUAGGUCCCUGAGACCCUUUAACCUGUGAGGACAUCCAGGGU |
| | | CACAGGUGAGGUUCUUGGGAGCCUGGCGUCUGGCC |
| 35 | miR-125b | UGCGCUCCUCUCAGUCCCUGAGACCCUAACUUGUGAUGUUUACCGUUUAAA |
| | | UCCACGGGUUAGGCUCUUGGGAGCUGCGAGUCGUGCU |
| 36 | miR-148a | GAGGCAAAGUUCUGAGACACUCCGACUCUGAGUAUGAUAGAAGUCAGUGCA |
| | | CUACAGAACUUUGUCUC |
| 37 | miR-16 | GUCAGCAGUGCCUUAGCAGCACGUAAAUAUUGGCGUUAAGAUUCUAAAAUU |
| | | AUCUCCAGUAUUAACUGUGCUGCUGAAGUAAGGUUGAC |
| 38 | miR-188 | UGCUCCCUCUCUCACAUCCCUUGCAUGGUGGAGGGUGAGCUUUCUGAAAAC |
| | | CCCUCCCACAUGCAGGGUUUGCAGGAUGGCGAGCC |
| 39 | snRNA-U6 | GTGCTCGCTTCGGCAGCACATATACTAAAATTGGAACGATACAGAGAAGAT |
| | | TAGCATGGCCCCTGCGCAA GGATGACACGCAAATTCGTGAAGCGTTCCAT |
| | | ATTTT |
| 40 | miR-7 | UUGGAUGUUGGCCUAGUUCUGUGUGGAAGACUAGUGAUUUUGUUGUUUUUA |
| | | GAUAACUAAAUCGACAACAAAUCACAGUCUGCCAUAUGGCACAGGCCAUGC |
| | | CUCUACAG |
| 41 | miR-92a | CUUUCUACACAGGUUGGGAUCGGUUGCAAUGCUGUGUUUCUGUAUGGUAUU |
| | | GCACUUGUCCCGGCCUGUUGAGUUUGG |
| 42 | miR-126 | CGCUGGCGACGGGACAUUAUUACUUUUGGUACGCGCUGUGACACUUCAAAC |
| | | UCGUACCGUGAGUAAUAAUGCGCCGUCCACGGCA |
| 43 | miR-25 | GGCCAGUGUUGAGAGGCGGAGACUUGGGCAAUUGCUGGACGCUGCCCUGGG |
| | | CAUUGCACUUGUCUCGGUCUGACAGUGCCGGCC |
| 44 | miR-152 | UGUCCCCCCCGGCCCAGGUUCUGUGAUACACUCCGACUCGGGCUCUGGAGC |
| | | AGUCAGUGCAUGACAGAACUUGGGCCCGGAAGGACC |
| 45 | miR-200a | CCGGGCCCCUGUGAGCAUCUUACCGGACAGUGCUGGAUUUCCCAGCUUGAC |
| | | UCUAACACUGUCUGGUAACGAUGUUCAAAGGUGACCCGC |
| 46 | miR-210 | ACCCGGCAGUGCCUCCAGGCGCAGGGCAGCCCCUGCCCACCGCACACUGCG |
| | | CUGCCCCAGACCCACUGUGCGUGUGACAGCGGCUGAUCUGUGCCUGGGCAG |
| | | CGCGACCC |
| 47 | miR-181a | AGAAGGGCUAUCAGGCCAGCCUUCAGAGGACUCCAAGGAACAUUCAACGCU |
| | | GUCGGUGAGUUUGGGAUUUGAAAAAACCACUGACCGUUGACUGUACCUUGG |
| | | GGUCCUUA |
| 48 | miR-30a-5p | GCGACUGUAAACAUCCUCGACUGGAAGCUGUGAAGCCACAGAUGGGCUUUC |
| | | AGUCGGAUGUUUGCAGCUGC |
| 49 | miR-34a | GGCCAGCUGUGAGUGUUUCUUUGGCAGUGUCUUAGCUGGUUGUUGUGAGCA |
| | | AUAGUAAGGAAGCAAUCAGCAAGUAUACUGCCCUAGAAGUGCUGCACGUUG |
| | | UGGGGCCC |
| 50 | miR-93 | CUGGGGGCUCCAAAGUGCUGUUCGUGCAGGUAGUGUGAUUACCCAACCUAC |
| | | UGCUGAGCUAGCACUUCCCGAGCCCCCGG |
| 51 | miR-326 | CUCAUCUGUCUGUUGGGCUGGAGGCAGGGCCUUUGUGAAGGCGGGUGGUGC |
| | | UCAGAUCGCCUCUGGGCCCUUCCUCCAGCCCCGAGGCGGAUUCA |

### Example Two: Islet Sodium Nitroprusside (SNP) Exposure Causes a Release of microRNAs

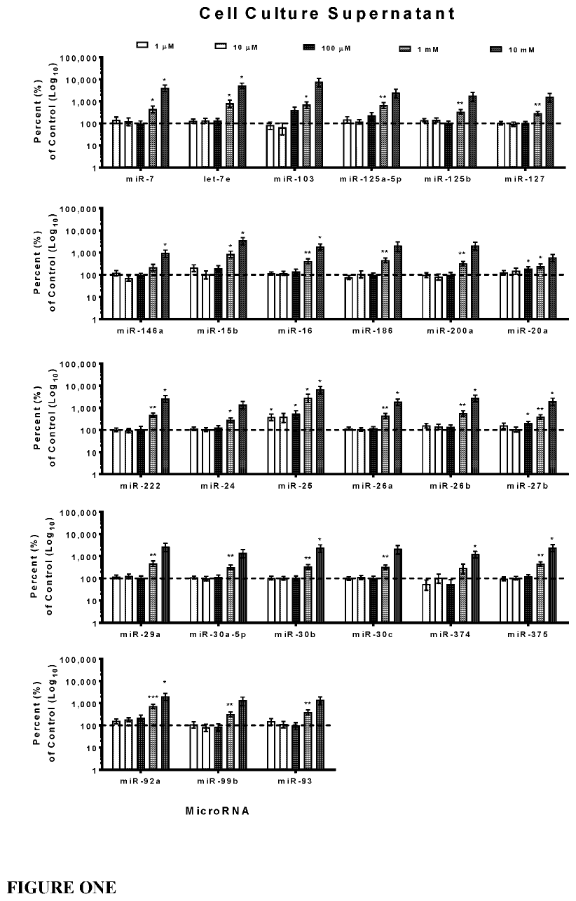

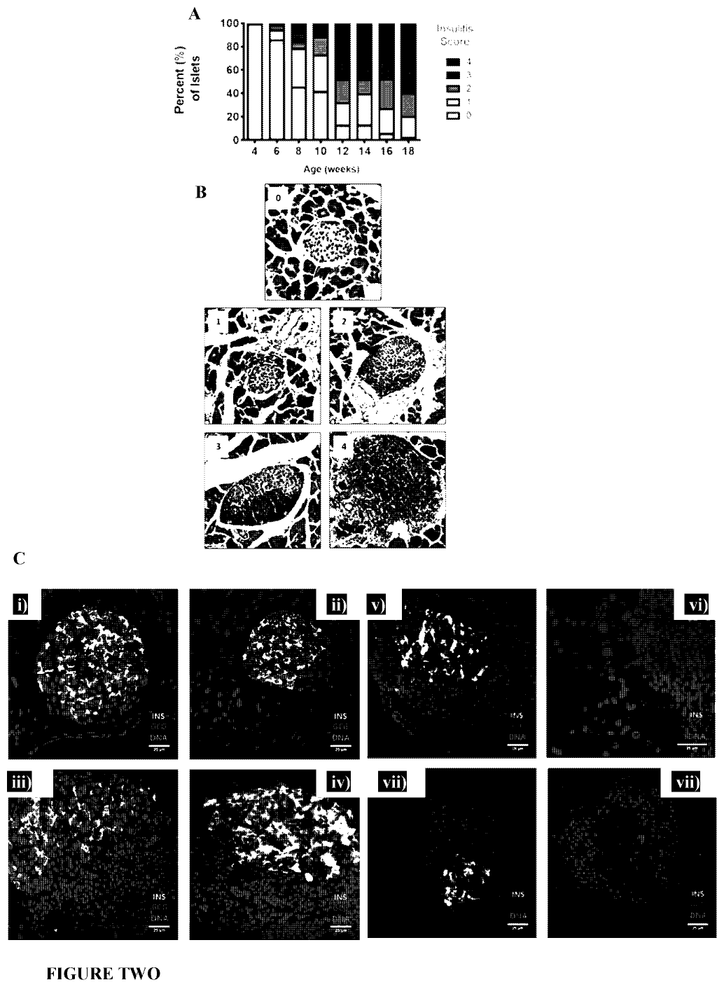

A total of 27 miRNAs were increased in the cell supernatant following a 24-hour exposure of human cadaveric islet preparations to sodium nitroprusside; a nitric oxide donor (FIG. 1). Data presented as Mean±SEM. * P<0.05 ** P<0.01 *** P<0.001 (t-test, deviation from 100%). N=6-7 different human cadaveric islet preparations for all concentrations apart from 10 mM (N=3 islet preps). Each islet preparation was conducted in multiple (3 or 4) technical replicates.

### Example Three: Insulitis in a Mouse Model of Type 1 Diabetes

Analyses of insulitis were made on a mouse model of type 1 diabetes (Non-obese diabetic (NOD) mice. These mice naturally develop type 1 diabetes and thus offer an opportunity to study the changes in microRNAs detected in serum/plasma of mice at risk of or progressing to type 1 diabetes. An aim of this study was to identify microRNA/s associated with progression to clinical diabetes as seen in human T1D.

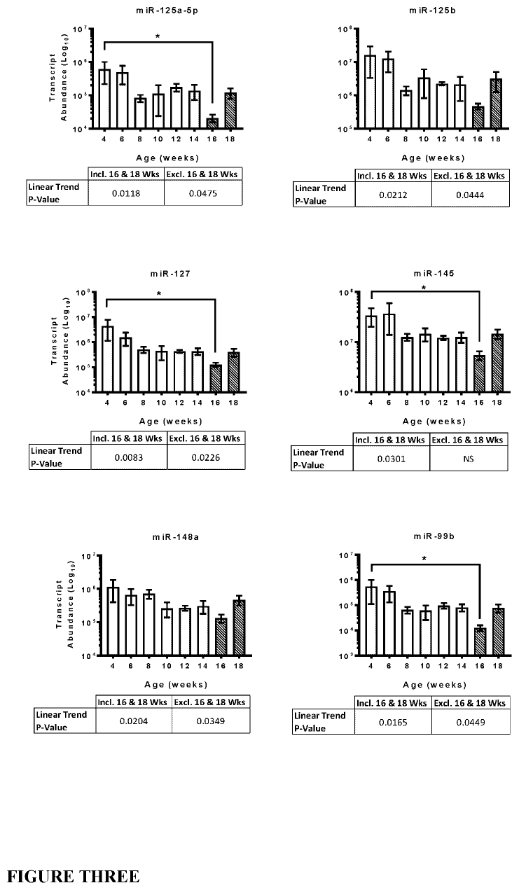

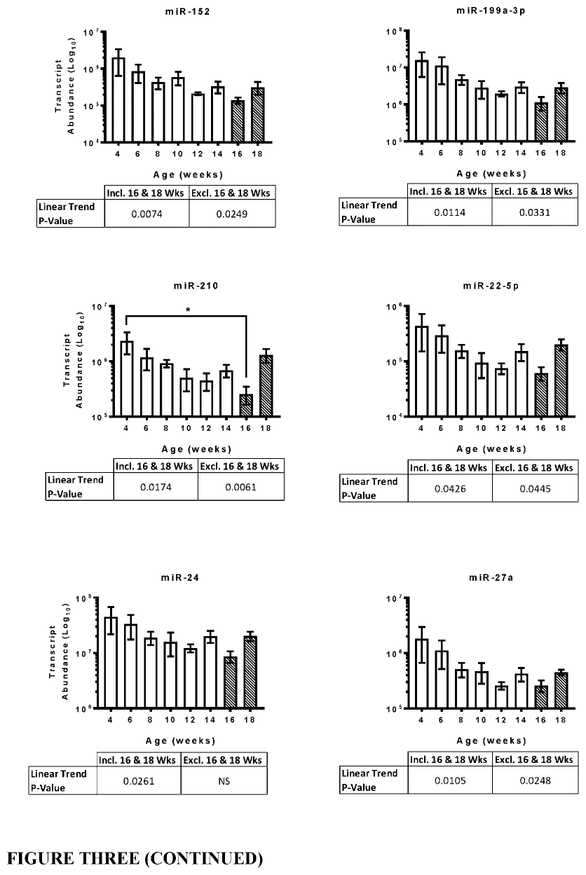

FIG. 2A shows the percentage of pancreatic islets present over an 18-week timecourse. Percentage of islets scored as 0 (no insulitis), 1 (peri-insulitis with up to 25% infiltration), 2 (25-50% infiltration), 3 (50-75% infiltration), or 4 (>75% infiltration). Percentages were based on H&E stained islets from four mice at each time point, with multiple layers being analysed.

Representative photographs of islets with insulitis scores of 0, 1, 2, 3 or 4. N=4 mice per time point are shown in FIG. 2B. Total islet numbers used for calculations=106 (4 wk), 95 (6 wk), 145 (8 wk), 133 (10 wk), 89 (12 wk), 64 (14 wk), 133 (16 wk), 106 (18 wk).

Representative fluorescent confocal images of insulin (green), glucagon (red) and DNA (blue) stained NOD mice islets at (i) 4, (ii) 6, (iii) 8, (iv) 10, (v) 12, (vi) 14, (vii) 16, and (viii) 18 weeks of age are shown in FIG. 2C.

### Example Four: Abundance of Circulating miRNAs in NOD Mice

Analyses of circulating miRNAs were made in (NOD) mice over time. As insulitis and the death of beta cells in the pancreas of these NOD mice is well characterised, the assessment of samples from very early on in their life helped in profiling all microRNAs that are differentially expressed in their circulation and could be potential biomarkers of diabetes progression.

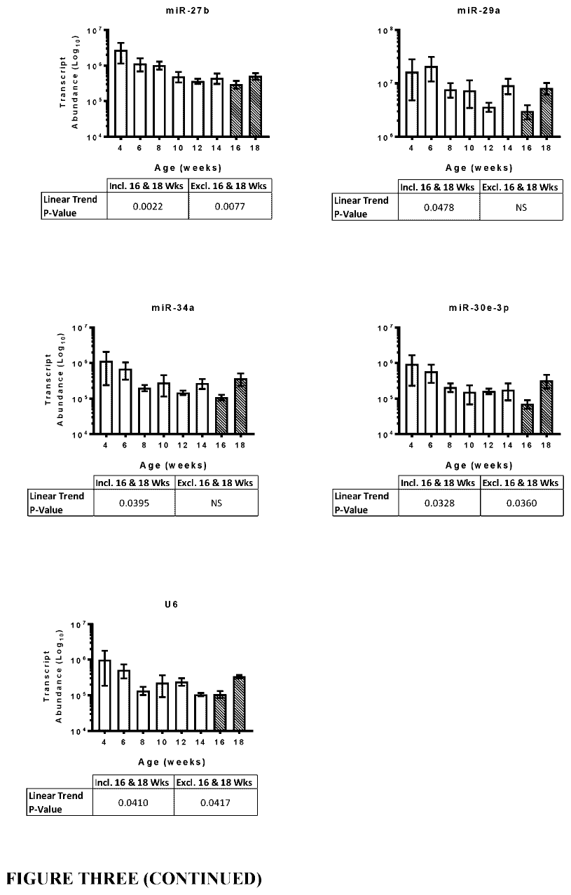

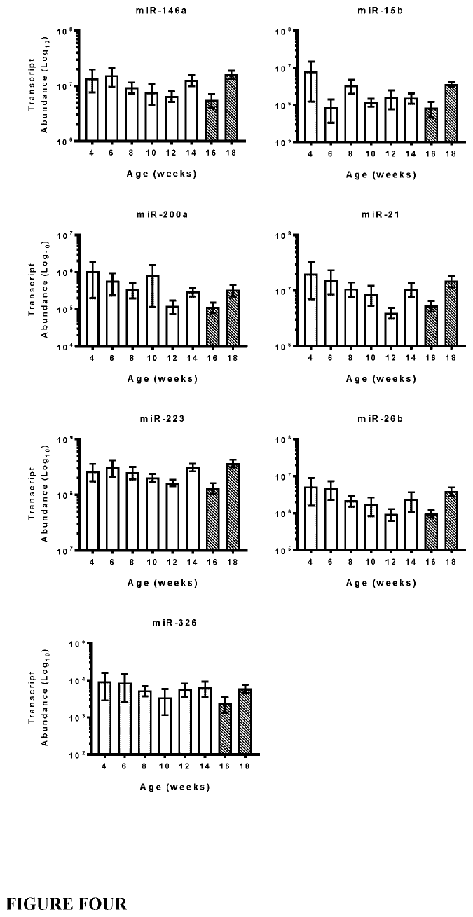

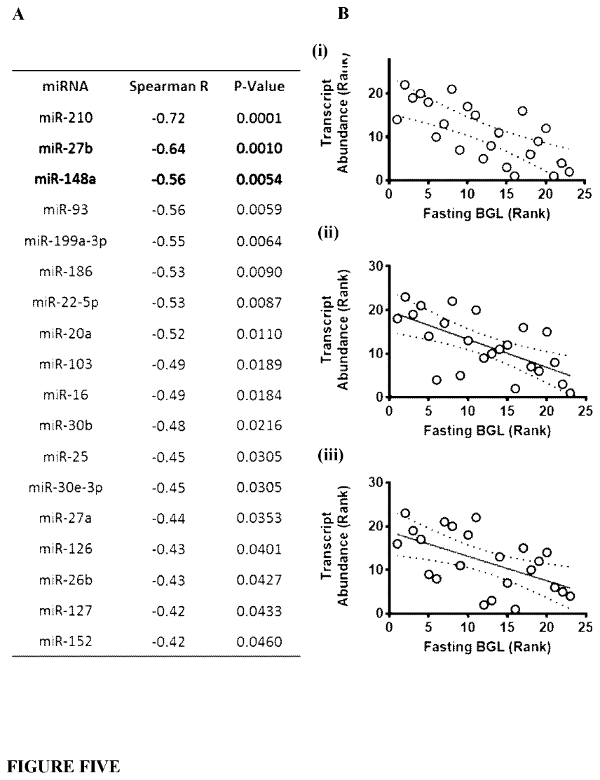

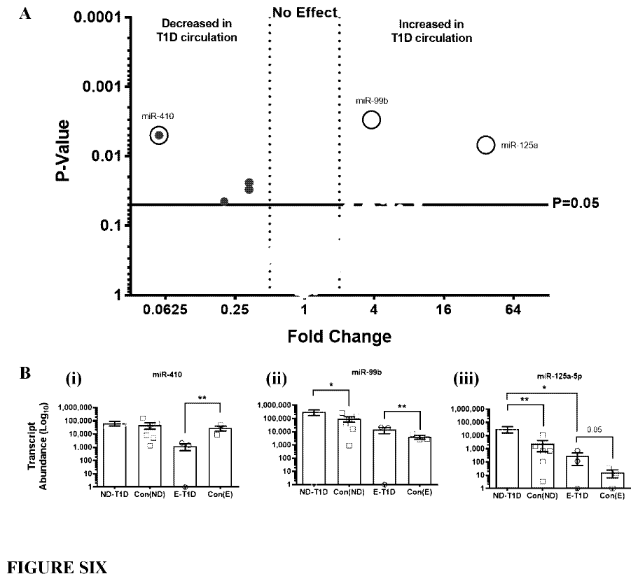

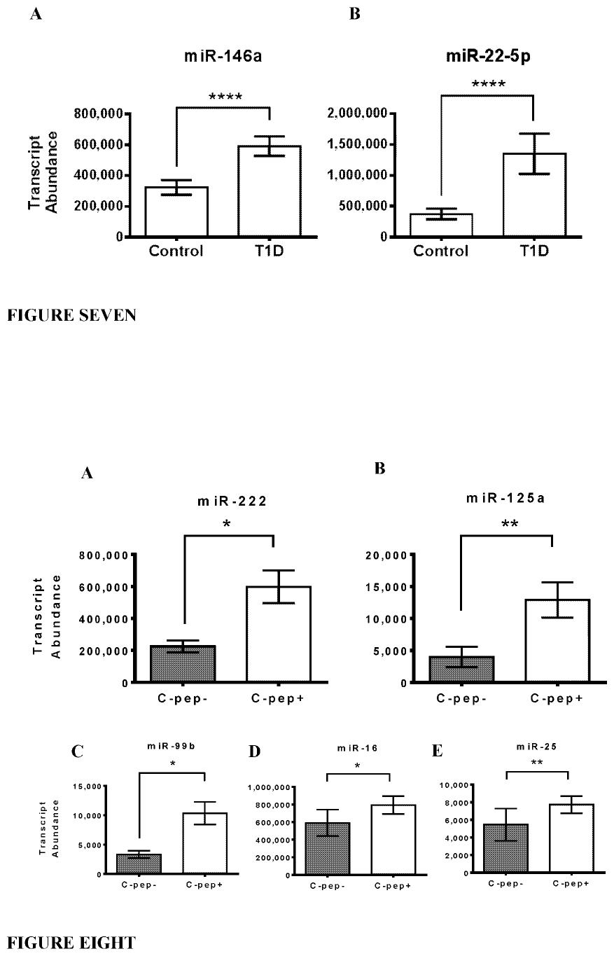

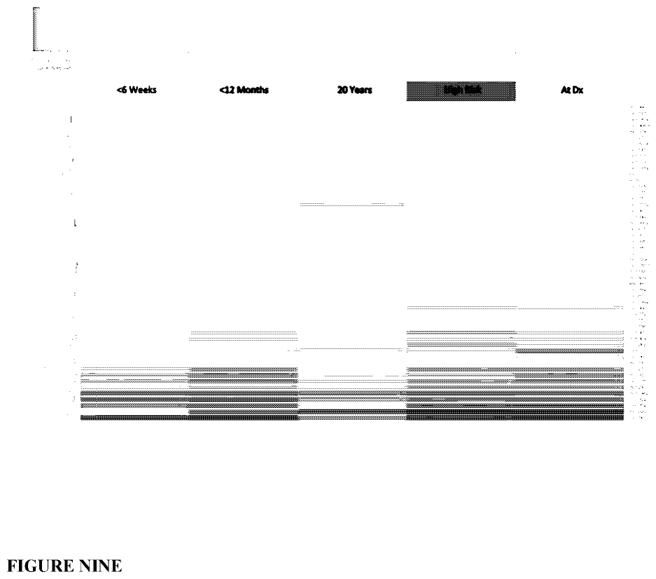

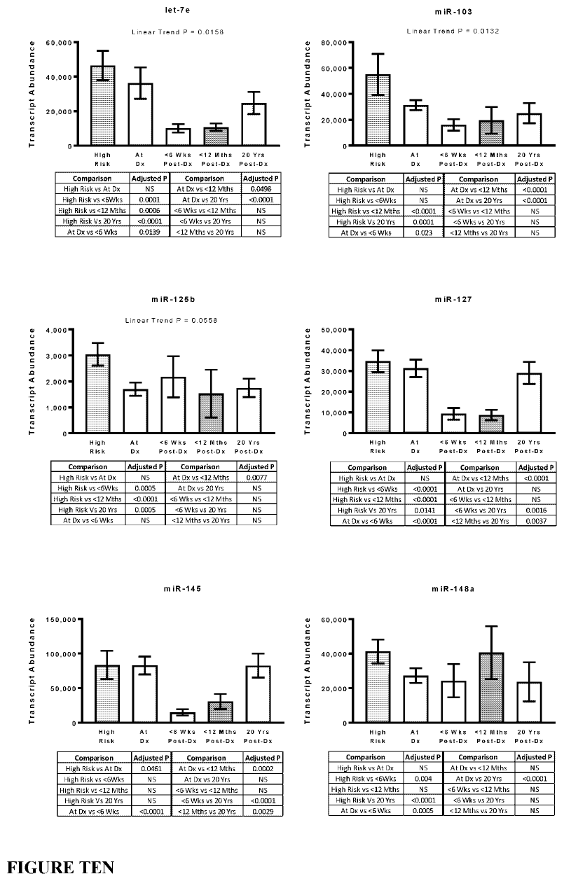

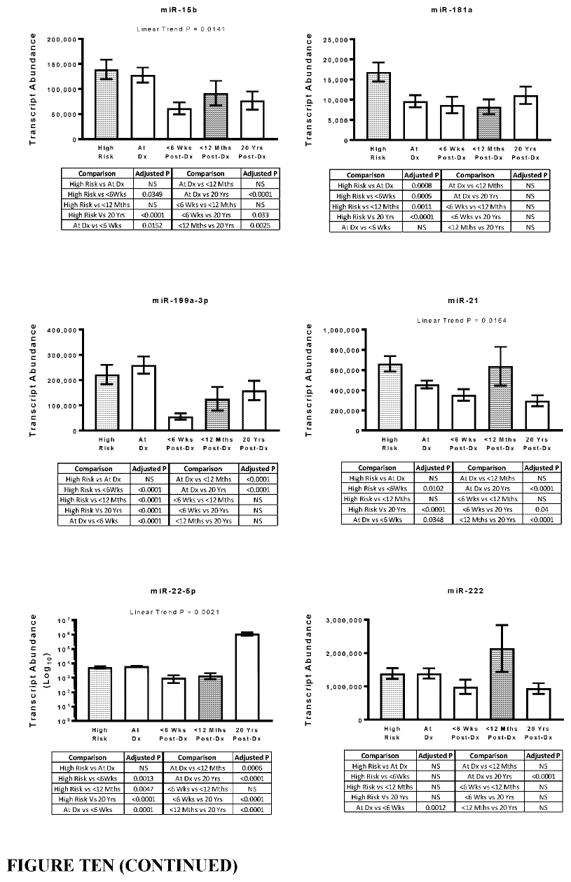

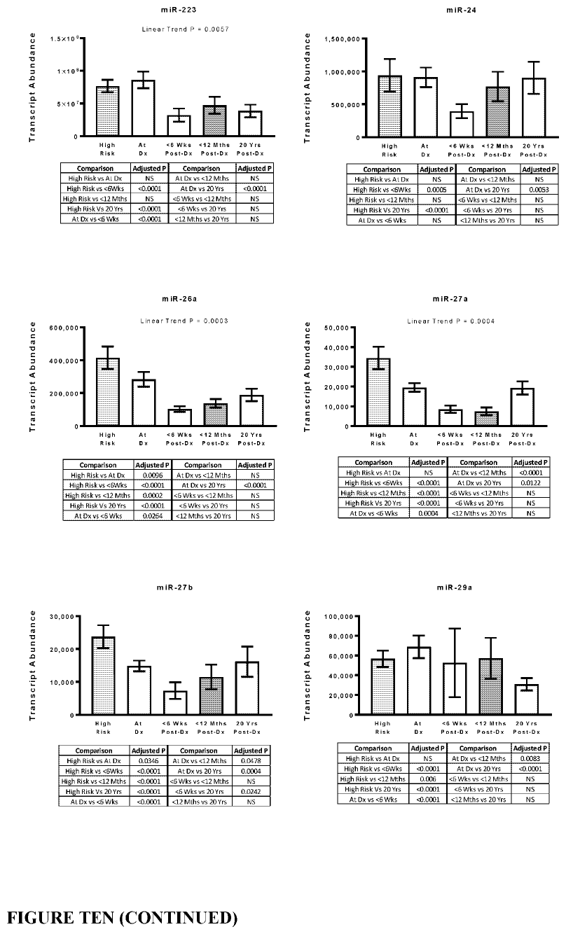

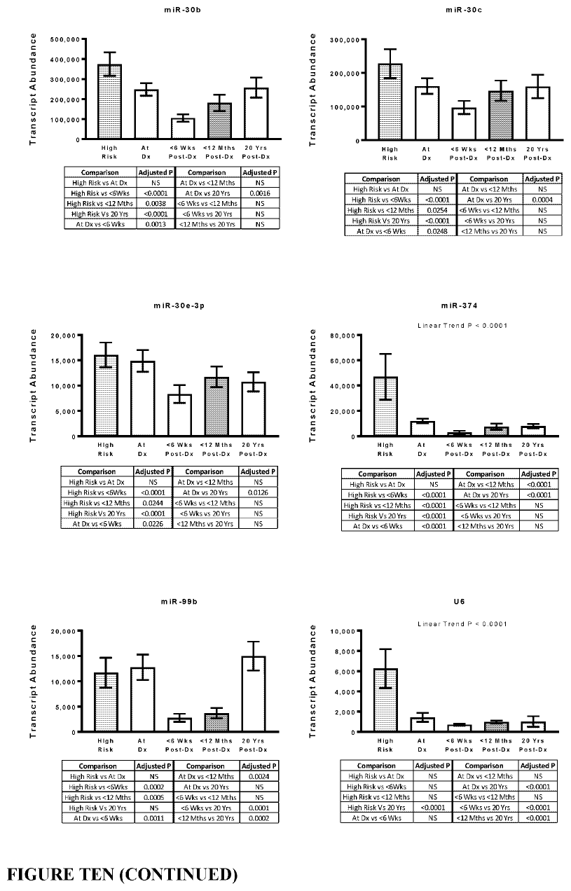

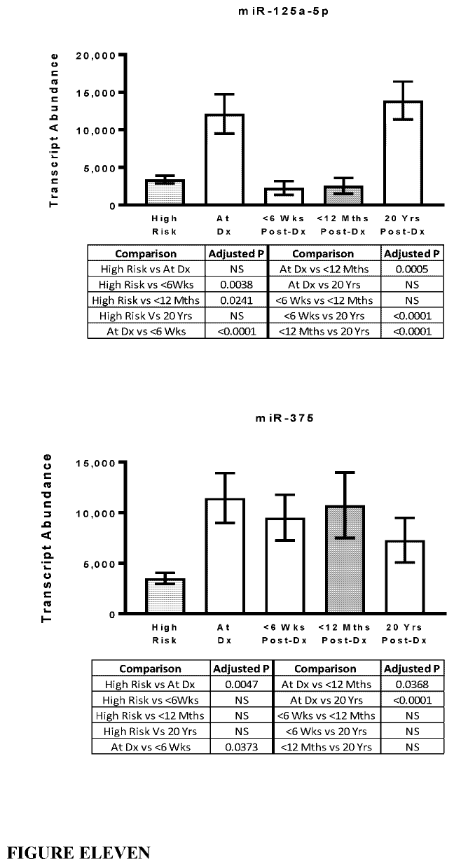

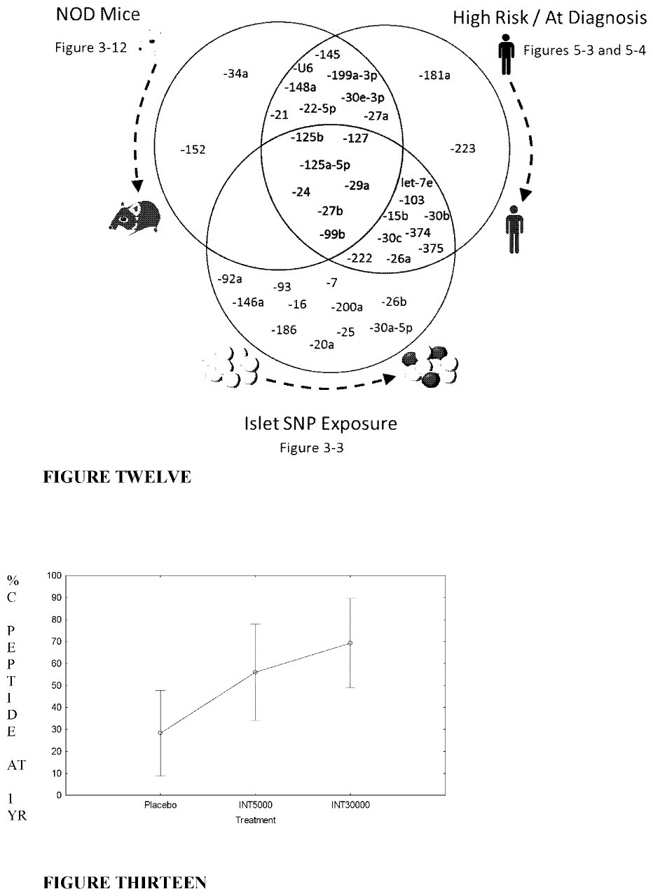

The abundance of some circulating miRNAs changes with age in NOD mice. As shown in FIG. 3 some circulating miRNAs decrease over time and significantly correlate with age in NOD mice (Mean±SEM. Linear trend, P<0.05 significant). In FIG. 3, Linear trend P-values are shown underneath each graph and results from 16 and 18 weeks of age are included (Incl. 16 & 18 Wks) or excluded (Excl. 16 & 18 Wks). Hatched bars are indicative of omitted groups. N=4 mice at each time point, except 4 weeks (N=3, see section 3.3.4). * P<0.05 (Kruskal-Wallis non-parametric ANOVA with corrected Dunn's multiple comparison test).

In contrast to those shown in FIG. 3, other circulating miRNAs were observed to be stable in NOD mice over time. Seven of the miRNA tested show no significant differences or trends between age groups of NOD mice (with or without omitted groups) (see FIG. 4). Mean±SEM. N=4 mice at each time point, except 4 weeks (N=3, see section 3.3.4). Hatched bars are indicative of groups omitted from other analyses.

### Example Five: Correlation Between Circulating microRNAs and Fasting Blood Glucose in NOD Mice

Circulating microRNAs demonstrated stronger correlation with fasting blood glucose in NOD mice (FIG. 5). 18 miRNAs were found to significantly correlate with the fasting BGLs in NOD mice (FIG. 5A). Three representative miRNAs were plotted against the respective fasting BGL using Spearman ranks (FIG. 5B parts i)-iii)). Data from 16 and 18-week old mice were omitted. Colours correspond to highlighted miRNAs in (FIG. 5A). The line of best fit (solid) with 95% confidence interval (dotted) is shown. N=27 mice. Spearman correlation, significance P<0.05. As a result of the animal colony issues during housing of week 16 and 18 animals, these mice did not show high blood glucose for both time points and hence were excluded. All data are cross-sectional.

### Example Six: Circulating microRNA Profiling of Patients with T1D

In order to compare the differences in circulating microRNA profiles of individuals without and with diabetes, microRNAs were assessed from established as well as new-onset T1D individuals, and the relative expression of microRNAs were analysed. Data were plotted in form of a volcano plot to identify potentially interesting and important microRNA candidates. Circulating microRNA profiles were generated from age- and gender-matched individuals without or with type 1 diabetes (T1D). The volcano plot of FIG. 6A shows differentially abundant miRNAs measured within the circulation of patients with newly-diagnosed (ND-T1D) and established (E-T1D) T1D, compared to age and gender-matched controls (Con(ND) and Con(E) respectively). Dotted line=2-fold change. FIG. 6B shows a subset of significant miRNAs highlighted in FIG. 6A with newly diagnosed T1D and controls in green and established T1D and controls in blue. Mean±SEM. N=9 T1D (6 ND-T1D, 3 E-T1D), 9 Control (6 ND-Con, 3 E-Con). * P<0.05, ** P<0.01.

### Example Seven: Elevated Circulating microRNAs in Patients with T1D

In order to assess the miRNA signature, levels of these miRNAs were quantified within the plasma of 180 individuals with established T1D and 138 age and gender matched controls. Two miRNAs were found to be significantly elevated in the circulation of individuals with established T1D compared to age and gender matched controls (FIGS. 7A and 7B). N=138 controls, 180 T1D. **** P<0.0001. Mean±SEM.

### Example Eight: Elevated Circulating microRNAs in T1D Patients with Detectable c-Peptide

Circulating C-peptide concentrations were measured as an indicator of residual β-cell function. Controls subjects had an average C-peptide concentration of 559.5±245.4 pM, while T1D subject had 29.3±87.6 pM (P<0.0001, unpaired t-test with Welch's correction). Due to the use of an ultra-sensitive ELISA, C-peptide was detectable in 123 of the T1D subjects. Five miRNAs were significantly elevated in the circulation of T1D patients with detectable C-peptide (C-pep+), compared to those without detectable C-peptide (C-pep−) (FIGS. 8A-8E). N=53 C-pep−, 123 C-pep+. Mean±SEM.

### Example Nine: miRNA Signature Expression During T1D Progression

Although longitudinal samples of individuals throughout their life course are highly desirable for such molecular biomarker discovery and validation, they are not available for research. These were therefore assessed in cross-sectional groups of samples of individuals at risk of T1 D (first-degree relatives of T1D individuals), clinically diagnosed T1D individuals, and in individuals up to −20 years following the clinical diagnosis of T1D. Features of these human sample sets used in the analysis of T1D progression are shown in Table 32 below.

TABLE 3

| | | At | <6 | 6 Weeks-12 | | |
|---|---|---|---|---|---|---|
| | High | Diagnosis | Weeks | Months | 20 Years | |
| | Risk | (Dx) | Post-Dx | Post-Dx | Post-Dx | P-Value |
| Cohort | 1 | 2 | 3A | 3B | 4 | — |
| (Section)^{a} | (5.3.1.1) | (5.3.1.2) | (5.3.1.3) | (5.3.1.3) | (5.3.1.4) | |
| Subjects, N | 19 | 199 | 57 | 19 | 220 | — |
| Time point | 3.5 ± 1.6 | 1 | 1 | 3 ± 1.2 | 1 | — |
| samples per | | | | | | |
| subject, N | | | | | | |
| (mean ± SD) | | | | | | |
| Females, % | 58 | 47 | 32 | 37 | 57 | 0.0047^{b} |
| Age, yrs | 7.0 ± 4.0 | 8.6 ± 4.1 | 10.1 ± 4.6 | 9.4 ± 4.0^{c} | 39.5 ± 13.5 | <0.0001^{d} |
| (mean ± SD) | | | | | | |
| Time since | — | 2 ± 1.4 | Up to 6 | Between | 20.2 ± 12.7 | — |
| diagnosis | | days | weeks | 6 weeks | years | |
| | | (mean ± SD) | | and 12 | | |
| | | | | months | | |
| HbA1c, % | NA | 11.6 ± 2.06 | 7.5 ± 1.2 | 6.97 ± 1.03 | 7.9 ± 1.3 | <0.0001^{d} |
| (mean ± SD) | | | | | | |
| HbA1c, | | 103.2 ± 22.5 | 58.3 ± 13.0 | 53.2 ± 11.2 | 63.3 ± 14.5 | <0.0001^{d} |
| mmol/mol | | | | | | |
| (mean ± SD) | | | | | | |

A heatmap of miRNA signature expression during T1D progression is shown in FIG. 9. Each square represents the mean value of the particular miRNA in each cohort. Complete linkage, Euclidean distance. N=75 (High Risk), 187 (At Dx), 54 (<6 Wks and <12 Mths Post-Dx), 218 (20 Yrs Post Dx).

FIG. 10 provides an analysis of the abundance of different microRNAs in individuals that are at high risk of T1D and in T1D individuals at diagnosis. It was observed that microRNAs are elevated in high-risk individuals and at T1D diagnosis. N=75 (High Risk), 187 (At Dx), 54 (<6 Wks and <12 Mths Post-Dx), 218 (20 Yrs Post-Dx). Mean±SEM. Multiple comparisons and adjusted P-values are listed in the table below each graph. Significant differences are highlighted. Linear trend significance P<0.05.

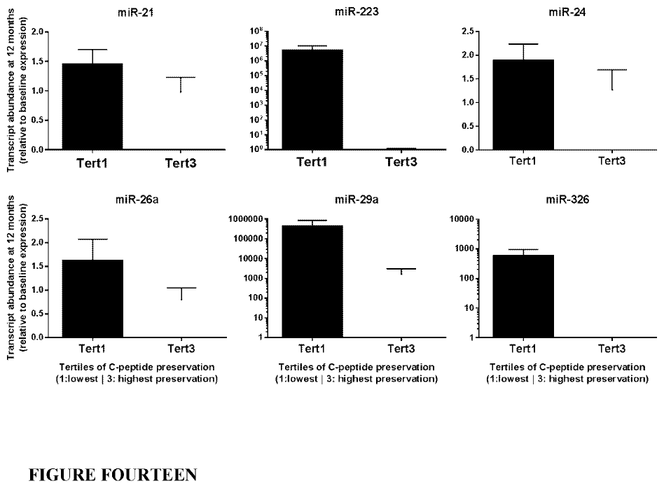

Two miRNAs were observed to be elevated at T1D diagnosis (FIG. 11). N=75 (High Risk), 187 (At Dx), 54 (<6 Wks and <12 Mths Post-Dx), 218 (20 Yrs Post-Dx). Mean±SEM. Multiple comparisons and adjusted P-values are listed in the table below each graph.

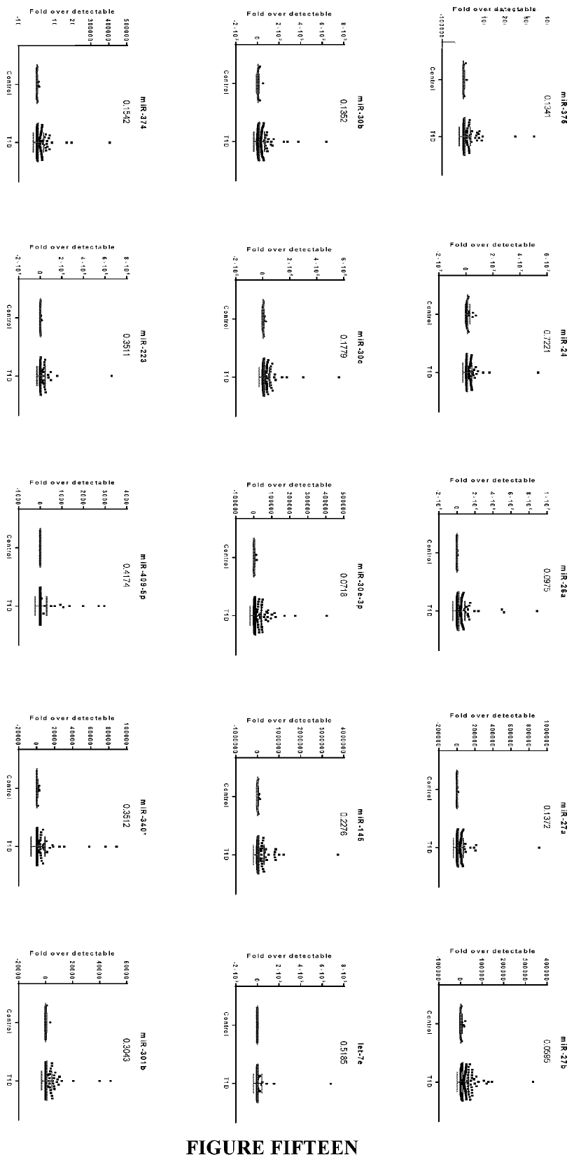

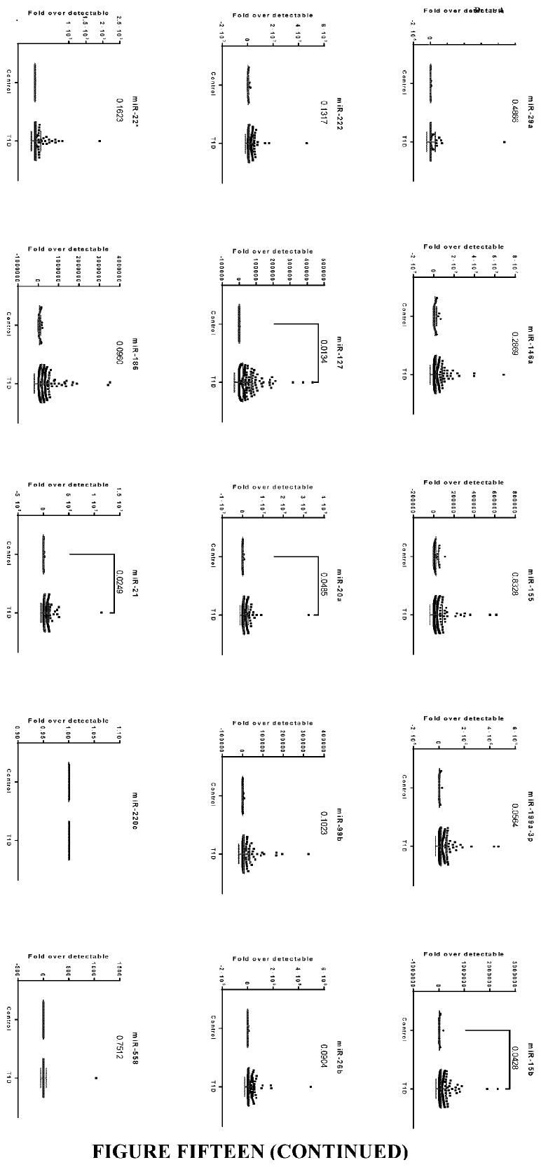

The Venn diagram of FIG. 12 shows an overview of the major findings in miRNA signature expression during T D progression. miRNAs found to be elevated in NOD mice (blue circle), released after human islet SNP exposure (yellow circle), elevated in individuals at high risk of T1D or at the point of diagnosis (red circle), or a combination of all three. MicroRNA-9, -126, -155, -188, -210, -220c, -301b, -326, -340-3p, -409-5p, -558, and -625 were not found to significantly change in any of these studies.

### Example Ten: Effect of Ingested Interferon-α on Residual C-Peptide

In order to assess whether microRNAs could predict treatment efficacy, microRNAs were profiled from a de-identified clinical study set of samples from new-onset T1D individuals. These individuals received oral interferon-α at two doses as a therapy to preserve beta cell mass. The effect of ingested interferon-α on residual C-peptide (a measure of beta cell mass) at one year following treatment with 5000 units, 30,000 units or placebo was investigated. The time points for IFNα or placebo treatment samples are shown in Table 4 below.

TABLE 4

| Time | Individuals, N | Days After Baseline |
|---|---|---|
| Point | (Placebo/5,000/30,000) | (mean ± SD) |
| T0 | 13/8/10 | 0 |
| T1 | 13/8/10 | 102.2 ± 17.3 |
| T2 | 13/8/10 | 203.2 ± 16.3 |
| T3 | 13/8/10 | 307.2 ± 22.4 |
| T4 | 13/8/10 | 420.5 ± 43.8 |

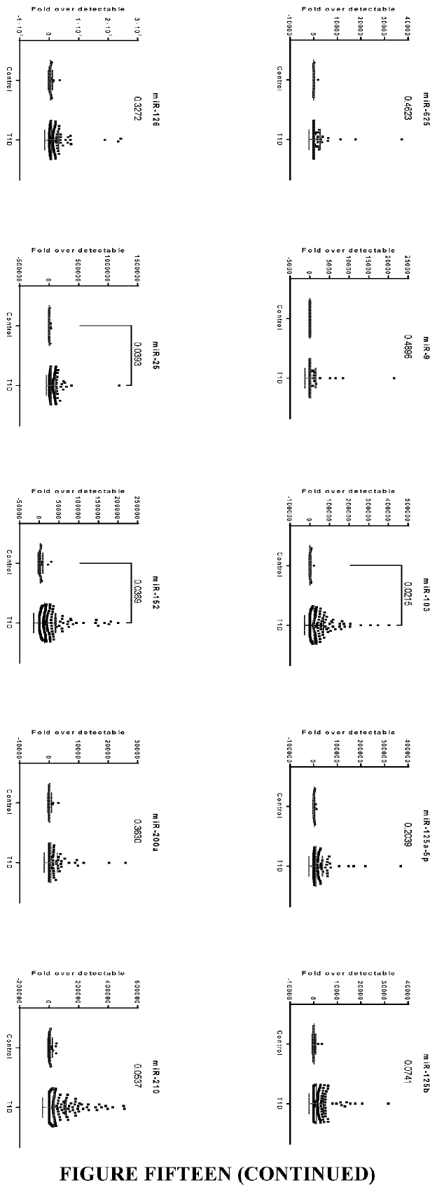

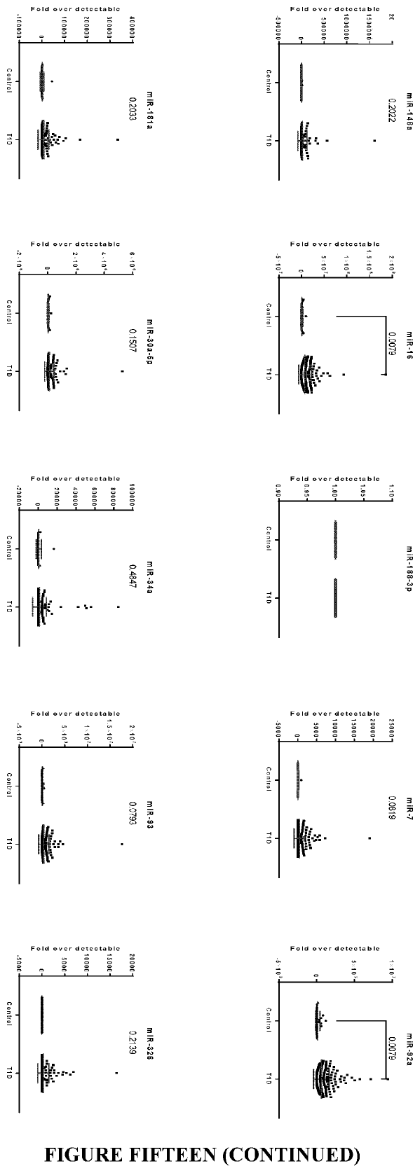

The results are shown in FIG. 13. Treatment groups are shown on the X-axis. The remaining beta-cell function measured as circulating C-peptide levels at 1 year (relative to baseline) are plotted on Y-axis (adjusted means, vertical bars denote 0.95 confidence intervals). A significant preservation of islet beta cell mass was seen in the 30,000 units ingested IFN treatment group as compared to the Control group. The miRNA signature of beta cell death (see summary FIG. 12) shows lower abundance indicating beta cell preservation in these individuals. These data indicate the potential use of this microRNA signature in assessing pancreatic beta cell death in response to a treatment that proffers the preservation of insulin-producing cells at 12 months from study baseline (N=34 individuals at baseline and at one year).

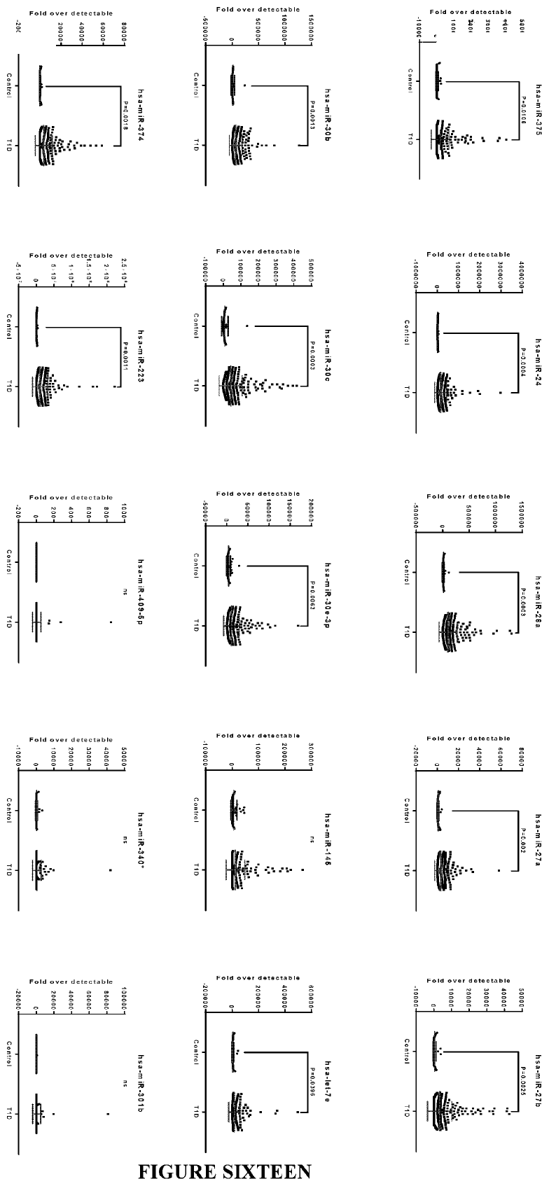

FIG. 14 demonstrates that the microRNA signature of beta cell death decreased in individuals showing the highest preservation of beta-cell mass at one year of interferon-α treatment. These data support the use of this microRNA signature for assessing treatment efficacy, as shown here with interferon-α in this case.

### Example Eleven: Circulating microRNA Signature Profiling in Ethnically Diverse Clinical Samples from Individuals with or without Type 1 Diabetes (TD1)

Part 1: Univariate Analyses

Real time PCR was carried out on 50 selected RAPID miRNAs on serum/plasma samples obtained from Control (healthy) individuals and individuals with Type 1 Diabetes (T1D). Cycle threshold (Ct) values were converted to fold over detectable (FoD), where Ct value of 39 was considered as limit of detection. Data were plotted as FoD values on Y axis for controls and T1D individuals and analyzed using two-tailed to unpaired t-test for each miRNA. P value for each comparison are presented.

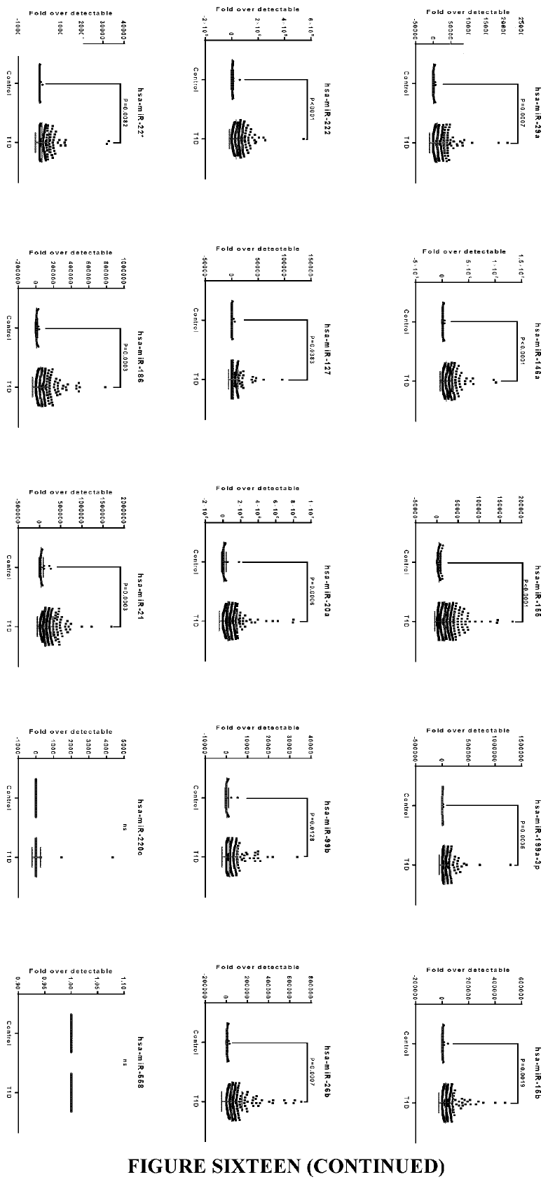

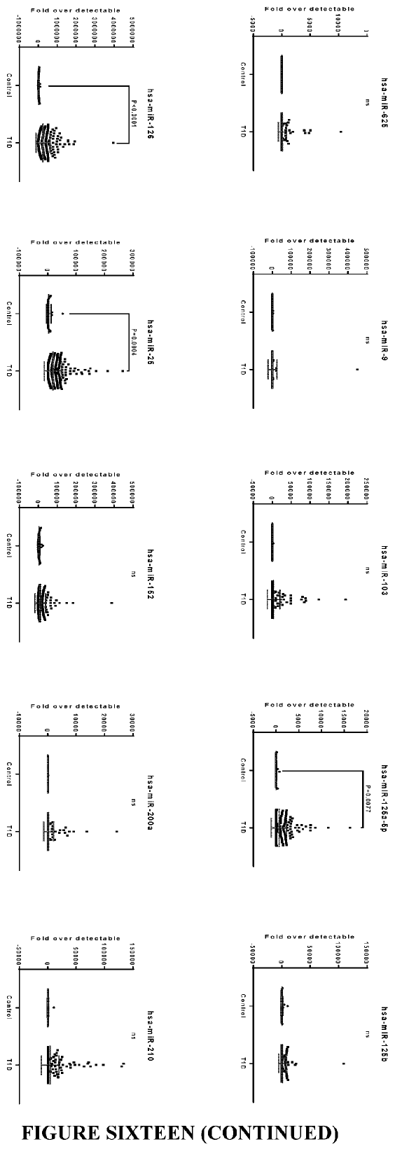

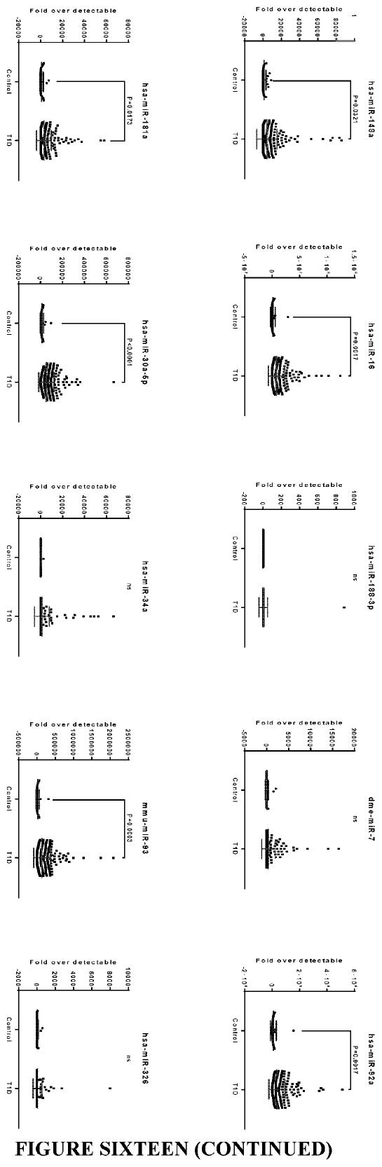

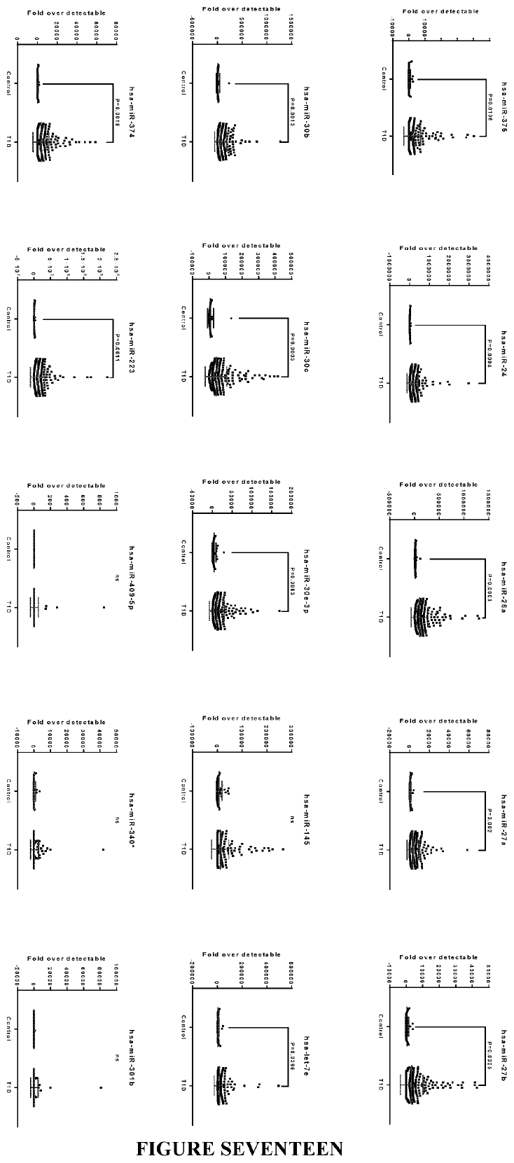

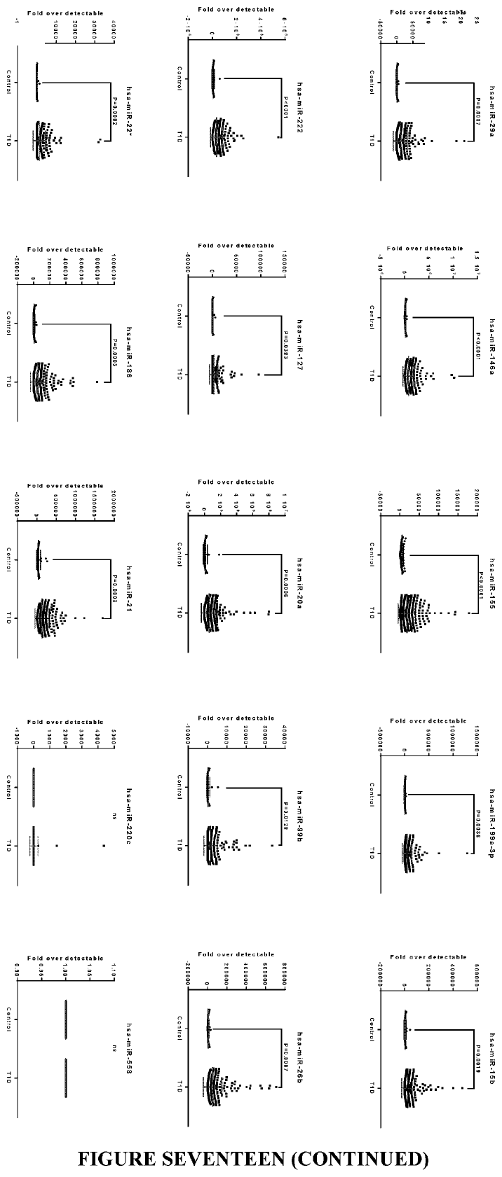

Study samples from three ethnically diverse populations were tested:
Australian individuals with T1D (n=337) or healthy controls (n=34)
Asian Indian individuals with T1D (n=270) or healthy controls (n=52)
Hong Kong Chinese individuals with T1D (n=120) or healthy controls (n=118) Results for the 50 miRNA panel in each population are shown in FIGS. 15-17.

Part 2: Random Forest Analysis

MicroRNAs associated with T1D status were analysed using the random forest analytical method (Kursa M B. “Robustness of Random Forest-based gene selection methods”. BMC Bioinformatics. 2014; 15:8. doi:10.1186/1471-2105-15-8), so as to identify a set of microRNAs that offer the highest predictive power for classifying individuals with T1D from those without T1D. Random forest is a supervised learning algorithm utilizing bootstrapping technique and decision tree modelling.

Briefly, the procedure works on the training set by randomly selecting features and calculating the node using best split point. The node is then split into “daughter” nodes using best split and the procedure is repeated until the target is reached as “leaf” node. All the above steps are repeated n times.

In the next step the features selected above are taken to the test set and used to predict the outcome (target, in this case dichotomous classification). For each target the votes are calculates and the high voted predicted target is forms the final prediction from random forest algorithm.

Importance of the features has been iteratively compared with the importance of so called “shadow” features created by shuffling the values of original cases. Attributes that have significantly worst importance than shadow ones are being consecutively dropped. On the other hand, attributes that are significantly better than shadows are selected as “important”. The y axis label Importance represents the Z-score of every feature in the shuffled dataset. Shadows are re-created in each iteration. Algorithm stops when only confirmed features are left.

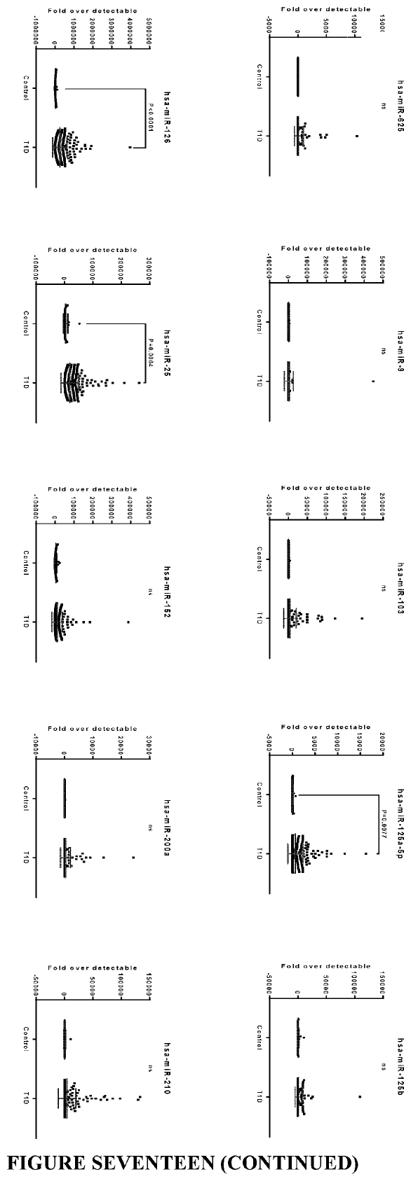

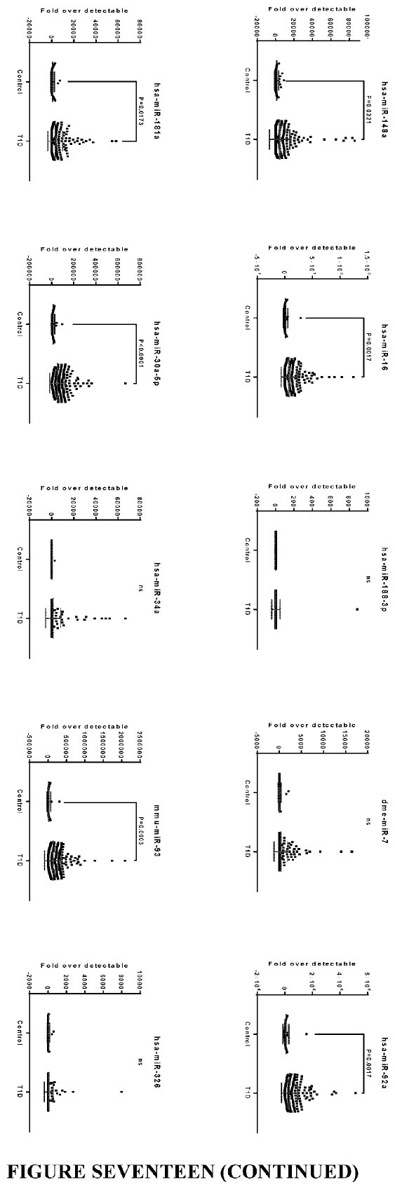

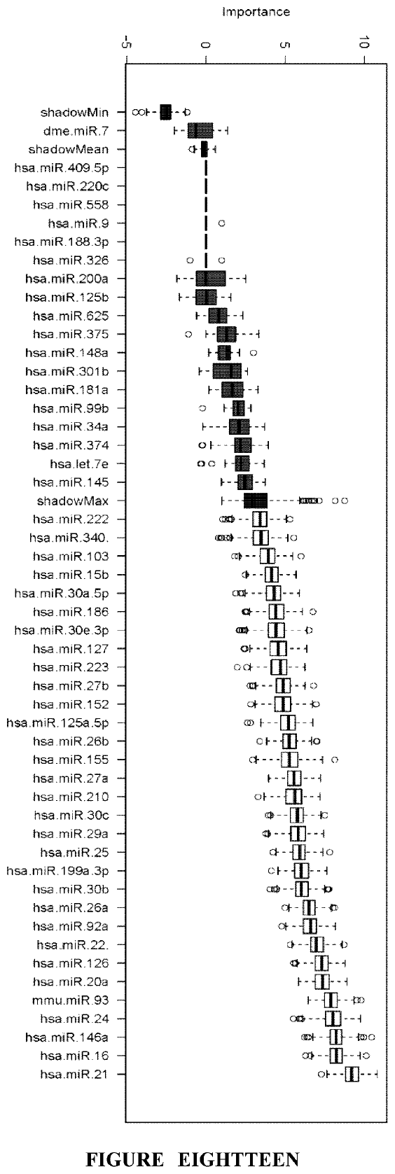

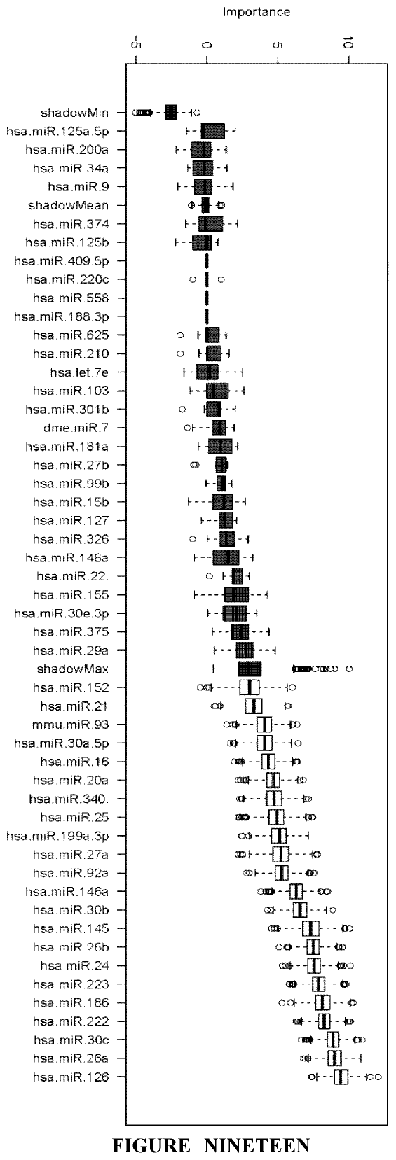

All the three datasets (from Australia, India and China) were analysed (see FIGS. 18-20). The Y-axis label represents the Z-score of every feature in the shuffled dataset. In this case, miRNAs are the features, which are shown on X-axis. MiRNAs with green plots are significantly better than cut-off/shadows (blue plots) and are considered as confirmed/important features to discriminate the control and T1D individuals in respective datasets. Blue plots in FIGS. 18-20 showing the minimum, mean and maximum importance obtained through the analysis of shadow features.

As summarised in Table 5, miRNAs capable of TD1 discrimination were in some cases common to 2 cohorts or all 3 cohorts.

TABLE 5

| TD1-discriminating | TD1-discriminating | TD1-discriminating |
|---|---|---|
| miRNAs present in all 3 | miRNAs present in 2 | miRNAs present in 1 |
| cohorts | cohorts | cohort |
| miR-16 | miR-15b | miR-7 |
| miR-20a | miR-22-5p | let-7e |
| miR-21 | miR-27a | miR-27b |
| miR-24 | miR-29a | miR-34a |
| miR-25 | miR-30e-3p | miR-103 |
| miR-26a | miR-126 | miR-125a-5p |
| miR-26b | miR-145 | miR-127 |
| miR-30a-5p | miR-155 | miR-148a |
| miR-30b | miR-340# | miR-152 |
| miR-30c | | miR-210 |
| miR-92a | | miR-374 |
| miR-93 | | miR-625 |
| miR-146a | | |
| miR-186 | | |
| miR-199a-3p | | |
| miR-222 | | |
| miR-223 | | |

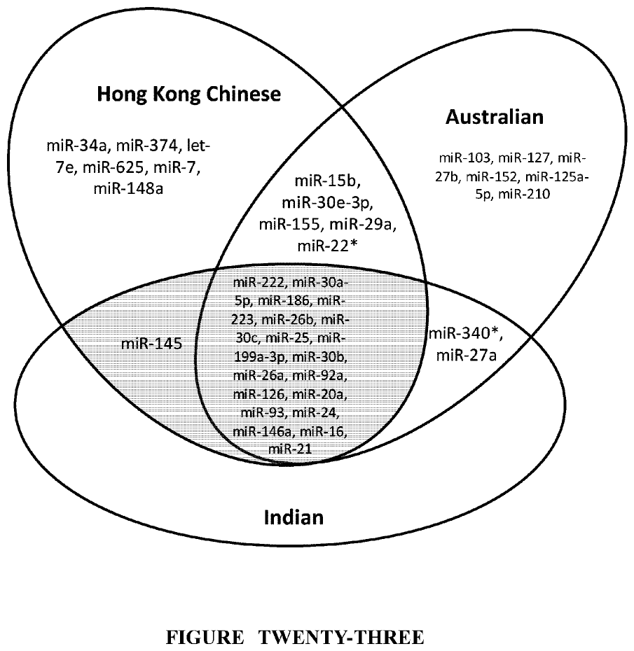

Table 6 and FIG. 23 summarise the distribution of discriminatory miRNAs in the three cohorts tested.

TABLE 6

| Australian Cohort | Indian Cohort | Chinese Cohort |
|---|---|---|
| | | miR-7 |
| | | let-7e |
| miR-15b | | miR-15b |
| miR-16 | miR-16 | miR-16 |
| miR-20a | miR-20a | miR-20a |
| miR-21 | miR-21 | miR-21 |
| miR-22-5p | | miR-22-5p |
| miR-24 | miR-24 | miR-24 |
| miR-25 | miR-25 | miR-25 |
| miR-26a | miR-26a | miR-26a |
| miR-26b | miR-26b | nnR-26b |
| miR-27a | miR-27a | |
| miR-27b | | |
| miR-29a | | miR-29a |
| miR-30a-5p | miR-30a-5p | miR-30a-5p |
| miR-30b | miR-30b | miR-30b |
| miR-30c | miR-30c | miR-30c |
| miR-30e-3p | | miR-30e-3p |
| | | miR-34a |
| miR-92a | miR-92a | miR-92a |
| miR-93 | miR-93 | miR-93 |
| miR-103 | | |
| miR-125a-5p | | |
| miR-126 | miR-126 | miR-126 |
| miR-127 | | |
| | mIR-145 | mIR-145 |
| miR-146a | miR-146a | miR-146a |
| | | miR-148a |
| miR-152 | | |
| miR-155 | | miR-155 |
| miR-186 | miR-186 | miR-186 |
| miR-199a-3p | miR-199a-3p | miR-199a-3p |
| miR-210 | | |
| miR-222 | miR-222 | miR-222 |
| miR-223 | miR-223 | miR-223 |
| miR-340# | miR-340# | |
| | | miR-374 |
| | | miR-625 |

Part 3: ROC Curve Analysis

Receiver operating characteristic (ROC) curves (Hajian-Tilaki K. “Receiver Operating Characteristic (ROC) Curve Analysis for Medical Diagnostic Test Evaluation”. Caspian Journal of Internal Medicine. 2013; 4(2):627-635) were generated for the Indian and Hong Kong cohorts using the important miRNAs (marked in green in FIG. 18) identified through the Australian cohort Random Forest analysis.

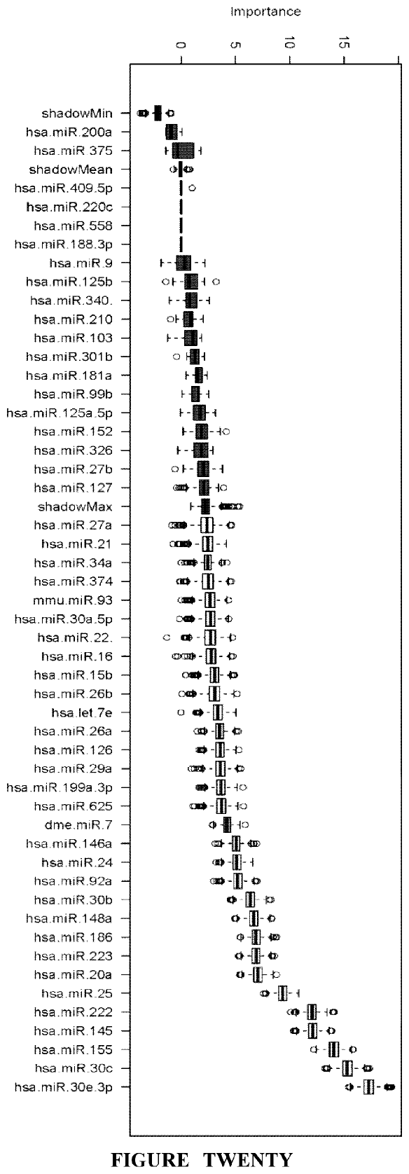

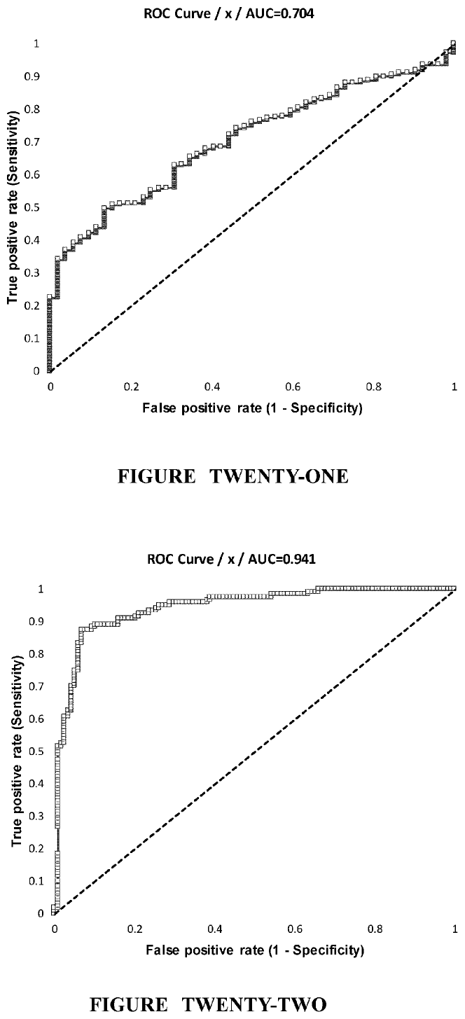

A total of 31 microRNAs were identified to be important in the Australian cohort. Of the 50 microRNAs in that panel, these 31 miRNAs provided the best model to predict T1D in the Asian Indian and HongKong Chinese datasets (FIGS. 22 and 23).

The area under the curve (AUC) for each cohort is presented at the top of each ROC curve (FIGS. 22 and 23), which represents the probability that a randomly chosen T1D subject from testing set is ranked correctly as having (or not having) Type 1 diabetes than a randomly chosen Control subject based on classification obtained from selected set of microRNAs. All analyses were carried by operators who were blinded to the sample identity.

1. A method for diagnosing, prognosing, or determining a likelihood of developing a disease or condition associated with or arising from reduced insulin production in a subject, the method comprising:
determining expression levels of ten or more extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject; and
comparing expression levels the microRNAs in the biological fluid sample to control expression level/s of the microRNAs generated from control biological fluid/s equivalent to the sample,
wherein elevated expression levels of the microRNAs in the biological fluid sample compared to the expression level/s of the microRNAs generated from the control biological fluid/s is indicative of a deficient or absent capacity for insulin production in beta-islet cells of the subject, and
the reduced or absent insulin production in the beta-islet cells is:
(i) predictive that the subject will develop the disease or condition; or
(ii) diagnostic of the disease or condition in the subject; or
(iii) prognostic that the disease or condition will progress in the subject.

2. A method for monitoring the response of a subject to a treatment for a disease or condition associated with or arising from reduced insulin production in a subject, the method comprising:
determining expression levels of ten or more extracellular microRNAs selected from the group consisting of: miR-24, miR-26a, miR-146a, mmiR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject; and
comparing expression levels the microRNAs in the biological fluid sample to control expression level/s of the microRNAs generated from control biological fluid/s equivalent to the sample,
wherein elevated expression levels of the microRNAs in the biological fluid sample compared to the expression level/s of the microRNAs generated from the control biological fluid/s is indicative of deficient or absent capacity for insulin production in beta islet cells of the subject, and a deficient or absent response to the treatment by the subject.

3. The method of claim 2, wherein the treatment comprises beta-islet cell transplantation, and/or the administration of a vaccine or therapeutic drug designed to retard loss of beta-islet cells in the subject and/or loss of insulin production capacity in beta-islet cells of the subject.

4. A method for assessing the efficacy of a treatment for inhibiting or preventing loss of beta-islet cell insulin production function and/or beta-islet cell death, the method comprising:
administering the treatment to a subject in need thereof,
determining expression levels of ten or more extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject; and
comparing expression levels the microRNAs in the biological fluid sample to control expression level/s of the microRNAs generated from control biological fluid/s equivalent to the sample,
wherein equivalent or reduced expression levels of the microRNAs in the biological fluid sample compared to the expression level/s of the microRNAs generated from the control biological fluid/s is indicative that the treatment has efficacy in inhibiting or preventing loss of beta-islet cell insulin production function and/or beta-islet cell death.

5. The method of any one of claims 1 to 4, further comprising removing any cells comprising nuclear material present in the biological fluid prior to determining expression levels of one or more extracellular microRNAs.

6. The method of claim 5, wherein said removing of the cells comprising nuclear material comprises lysing less than: 10%, 5%, 4%, 3%, 2%, 1%, 0.5%, 0.2% or 0.1% of the cells.

7. The method of any one of claims 1 to 6, wherein the condition or disease is selected from any of diabetes, pancreatitis, insulinoma, and pancreatic cancer.

8. The method of claim 7, wherein the disease is diabetes.

9. The method of claim 8, wherein the diabetes is Type 1 diabetes.

10. The method of any one of claims 1 to 9, wherein the biological fluid sample is whole blood.

11. The method of any one of claims 1 to 9, wherein the biological fluid sample is serum or plasma.

12. The method of any one of claims 1 to 11, further comprising an initial step of obtaining the biological fluid sample from the subject.

13. The method of any one of claims 1 to 12, wherein the ten or more microRNAs comprise or consist of any: 10, 11, 12, 13, 14, 15, 16, 17, or 18; of said microRNAs.

14. The method of any one of claims 1 to 13, wherein the subject is of Australian ethnicity, of Chinese ethnicity, or of Indian ethnicity.

15. The method of any one of claims 1 to 14, wherein the ten or more microRNAs are selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

16. The method of claim 15, wherein the microRNAs comprise or consist of any: 10, 11, 12, 13, 14, 15, 16, 17, 18, 19, 20, 21, 22, or 23; of said microRNAs.

17. The method of claim 15 or claim 16, wherein the subject is of Australian ethnicity or of Chinese ethnicity.

18. The method of any one of claims 1 to 13, wherein the ten or more microRNAs are selected from the group consisting of: miR-16, miR-20a, miR-21, miR-24, miR-25, miR-26a, miR-26b, miR-27a, miR-30a-5p, miR-30b, miR-30c, miR-92a, miR-93, miR-126, miR-146a, miR-186, miR-199a-3p, miR-222, miR-223, and miR-340.

19. The method of claim 18, wherein the microRNAs comprise or consist of any: 10, 11, 12, 13, 14, 15, 16, 17, 18, 19, or 20; of said microRNAs.

20. The method of claim 18 or claim 19, wherein the subject is of Australian ethnicity or of Indian ethnicity.

21. The method of any one of claims 1 to 13, wherein the ten or more microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

22. The method of claim 21, wherein the microRNAs comprise or consist of any: 10, 11, 12, 13, 14, 15, 16, 17, 18, or 19; of said microRNAs.

23. The method of claim 21 or claim 22, wherein the subject is of Chinese ethnicity or of Indian ethnicity.

24. The method of any one of claims 1 to 13, wherein the ten or more microRNAs are selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223

25. The method of claim 24, wherein the microRNAs comprise or consist of any: 10, 11, 12, 13, 14, 15, 16, 17, 18, 19, 20, 21, 22, 23, 24, 25, 26, 27, 28 or 29; of said microRNAs.

26. The method of claim 24 or claim 25, wherein the subject is of Australian ethnicity.

27. The method of any one of claims 1 to 13, wherein the ten or more microRNAs are selected from the group consisting of: let-7e, miR-7, miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

28. The method of claim 27, wherein the microRNAs comprise or consist of any: 10, 11, 12, 13, 14, 15, 16, 17, 18, 19, 20, 21, 22, 23, 24, 25, 26, 27, 28 or 29; of said microRNAs.

29. The method of claim 27 or claim 28, wherein the subject is of Chinese ethnicity.

30. The method of any one of claims 1 to 29, wherein the control biological fluid/s is/are obtained from control subject/s determined not to have a disease or condition associated with aberrant insulin production and/or abnormal insulin metabolism.

31. The method of claim 30, wherein the control subject/s are a population of individuals determined not to have a disease or condition associated with aberrant insulin production and/or abnormal insulin metabolism.

32. The method of claim 31 or claim 32, wherein the control subject/s do not have a disease or condition selected from the group consisting of diabetes, pancreatitis, insulinoma, and pancreatic cancer.

33. A kit comprising primers, probes and/or other binding agents for use in detecting expression of at least ten microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from the subject.

34. A microRNA signature comprising least ten microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93.

35. The kit of claim 33 or the microRNA signature of claim 34, wherein the at least ten microRNAs comprise or consist of any: 10, 11, 12, 13, 14, 15, 16, 17, or 18; of said microRNAs.

36. The kit of claim 33 or claim 35, or the microRNA signature of claim 34 or claim 35, wherein the at least ten microRNAs are selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

37. The kit or microRNA signature of claim 36, wherein the microRNAs comprise or consist of any: 10, 11, 12, 13, 14, 15, 16, 17, 18, 19, 20, 21, 22, or 23; of said microRNAs.

38. The kit of claim 33 or claim 35, or the microRNA signature of claim 34 or claim 35, wherein the at least ten microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-146a, miR-186, miR-199a-3p, miR-222, miR-223, miR-340 and miR-27a.

39. The kit or microRNA signature of claim 38, wherein the microRNAs comprise or consist of any: 10, 11, 12, 13, 14, 15, 16, 17, 18, 19, or 20; of said microRNAs.

40. The kit of claim 33 or claim 35, or the microRNA signature of claim 34 or claim 35, wherein the at least ten microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

41. The kit or microRNA signature of claim 40, wherein the microRNAs comprise or consist of any: 10, 11, 12, 13, 14, 15, 16, 17, 18, or 19; of said microRNAs.

42. The kit of claim 33 or claim 35, or the microRNA signature of claim 34 or claim 35, wherein the at least ten microRNAs are selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223.

43. The kit or microRNA signature of claim 42, wherein the microRNAs comprise or consist of any: 10, 11, 12, 13, 14, 15, 16, 17, 18, 19, 20, 21, 22, 23, 24, 25, 26, 27, 28 or 29; of said microRNAs.

44. The kit of claim 33 or claim 35, or the microRNA signature of claim 34 or claim 35, wherein the at least ten microRNAs are selected from the group consisting of: let-7e, miR-7, miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

45. The kit or microRNA signature of claim 44, wherein the microRNAs comprise or consist of any: 10, 11, 12, 13, 14, 15, 16, 17, 18, 19, 20, 21, 22, 23, 24, 25, 26, 27, 28 or 29; of said microRNAs.

46. The kit of any one of claims 33 or 35 to 45, further comprising any one or more of:
(i) one or more reagents for extracting the biological fluid sample;
(ii) one or more reagents for reverse-transcribing RNA into cDNA;
(iii) one or more reagents for amplifying the cDNA;
(iv) control standards for normalisation of microRNA signature expression data and/or comparison of microRNA signature expression data to determine whether expression of the microRNA signature is increased, reduced, and/or within a standard range;
(v) any one of more of: buffers, washing reagents, RNAse inhibitors, precipitators (e.g. glycogen), nuclease-free water, salt solutions.

47. Use of the kit of any one of claims 33 or 35 to 46, or the microRNA signature of any one of claims claim 34 to 46, for diagnosing, prognosing, or determining a likelihood of developing a disease or condition associated with or arising from reduced insulin production in a subject, wherein the disease or condition is selected from the group consisting of diabetes, pancreatitis, insulinoma, and pancreatic cancer.

48. Use of one or more agents for determining expression levels of ten or more extracellular microRNAs selected from the group consisting of: miR-24, miR-26a, miR-146a, miR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from a subject, for the preparation of a medicament for diagnosing, prognosing, or determining a likelihood of developing of a disease or condition associated with or arising from reduced insulin production in the subject.

49. Use of one or more agents for determining expression levels of ten or more extracellular microRNAs selected from the group consisting of: miR-24, miR-26a, miR-146a, mmiR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from a subject, for the preparation of a medicament for monitoring the response of the subject to a treatment for a disease or condition associated with reduced insulin production.

50. The use of claim 49, wherein the treatment comprises beta-islet cell transplantation, and/or the administration of a vaccine or therapeutic drug designed to retard loss of beta-islet cells in the subject and/or loss of insulin production capacity in beta-islet cells of the subject.

51. Use of one or more agents for determining expression levels of ten or more extracellular microRNAs selected from the group consisting of: miR-24, miR-26a, miR-146a, mmiR-199a-3p, miR-30b, miR-30c, miR-222, miR-20a, miR-26b, miR-223, miR-186, miR-21, miR-16, miR-92a, miR-126, miR-25, miR-30a-5p, and miR-93; in a biological fluid sample obtained from a subject, for the preparation of a medicament for assessing the efficacy of a treatment for inhibiting or preventing loss of beta-islet cell insulin production function and/or beta-islet cell death in the subject.

52. The use of any one of claims 48 to 51, wherein the microRNAs comprise or consist of any: 10, 11, 12, 13, 14, 15, 16, 17, or 18; of said microRNAs.

53. The use of any one of claims 48 to 52, wherein the subject is of Australian ethnicity, of Chinese ethnicity, or of Indian ethnicity.

54. The use of any one of claims 48 to 53, wherein the microRNAs are selected from the group consisting of: miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-92a, miR-93, miR-126, miR-146a, miR-155, miR-186, miR-199a-3p, miR-222, and miR-223.

55. The use of claim 54, wherein the microRNAs comprise or consist of any: 10, 11, 12, 13, 14, 15, 16, 17, 18, 19, 20, 21, 22, or 23; of said microRNAs.

56. The use of claim 54 or claim 55, wherein the subject is of Australian ethnicity or of Chinese ethnicity.

57. The use of any one of claims 48 to 53, wherein the ten or more microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-146a, miR-186, miR-199a-3p, miR-222, miR-223, miR-340 and miR-27a.

58. The use of claim 57, wherein the microRNAs comprise or consist of any: 10, 11, 12, 13, 14, 15, 16, 17, 18, 19, or 20; of said microRNAs.

59. The use of claim 57 or claim 58, wherein the subject is of Australian ethnicity or of Indian ethnicity.

60. The use of of any one of claim 48 to 53, wherein the ten or more microRNAs are selected from the group consisting of: miR-16, miR-20a, miR-21, miR-24, miR-25, miR-26a, miR-26b, miR-30a-5p, miR-30b, miR-30c, miR-92a, miR-93, miR-126, miR-145, miR-146a, miR-186, miR-199a-3p, miR-222, and miR-223.

61. The use of claim 60, wherein the microRNAs comprise or consist of any: 10, 11, 12, 13, 14, 15, 16, 17, 18, or 19; of said microRNAs.

62. The use of claim 60 or claim 61, wherein the subject is of Chinese ethnicity or of Indian ethnicity.

63. The use of any one of claims 48 to 53, wherein the ten or more microRNAs are selected from the group consisting of: miR-15b, miR-16, miR-20a, miR-21, miR-22-5p, miR-24, miR-25, miR-26a, miR-26b, miR-27b, miR-29a, miR-30a-5p, miR-30b, miR-30c, miR-30e-3p, miR-92a, miR-93, miR-103, miR-126, miR-125a-5p, miR-127, miR-146a, miR-152, miR-155, miR-186, miR-199a-3p, miR-210, miR-222, and miR-223

64. The use of claim 63, wherein the microRNAs comprise or consist of any: 10, 11, 12, 13, 14, 15, 16, 17, 18, 19, 20, 21, 22, 23, 24, 25, 26, 27, 28 or 29; of said microRNAs.

65. The use of claim 63 or claim 64, wherein the subject is of Australian ethnicity.

66. The use of any one of claims 48 to 53, wherein the ten or more microRNAs are selected from the group consisting of: let-7e, miR-7, miR-15b, miR-16, miR-20a, miR-21, miR-22-5p, miR-24, miR-25, miR-26a, miR-26b, miR-29a, miR-30a-5p, miR-30b, miR-30c, miR-30e-3p, miR-34a, miR-92a, miR-93, miR-126, miR-146a, mIR-148a, miR-155, miR-186, miR-199a-3p, miR-222, miR-223, miR-374, and miR-625.

67. The use of claim 66, wherein the microRNAs comprise or consist of any: 10, 11, 12, 13, 14, 15, 16, 17, 18, 19, 20, 21, 22, 23, 24, 25, 26, 27, 28 or 29; of said microRNAs.

68. The use of claim 66 or claim 67, wherein the subject is of Chinese ethnicity.
